(19) United States
(12) Patent Application Publication
(10) Pub. No.: US 2026/0337139 A1
(43) Pub. Date: Oct. 29, 2026

(54) IMPROVED PROCESS FOR THE PREPARATION OF 3,7-DIMETHYL-OCTA-2,6-DIENAL

(71) Applicant: BASF SE, Ludwigshafen am Rhein (DE)

(72) Inventors: Bernhard BRUNNER, Ludwigshafen am Rhein (DE); Gunnar HEYDRICH, Ludwigshafen am Rhein (DE); Rupert WAGNER, Ludwigshafen am Rhein (DE); Martin KAMASZ, Ludwigshafen am Rhein (DE)

(21) Appl. No.: 19/115,825

(22) PCT Filed: Sep. 28, 2023
(86) PCT No.: PCT/EP2023/076919

§ 371 Date: Mar. 27, 2025

(30) Foreign Application Priority Data
Sep. 28, 2022 (EP) 22198358.8

(51) Int. Cl. C07C 45/51

(52) U.S. Cl. CPC C07C 45/513; C07C 45/515

(57) ABSTRACT

A process for the preparation of 3,7-dimethyl-octa-2,6-dienal (citral) comprises the steps of a) continuously condensing prenol with prenal in the presence of at least one catalyst in a reaction column with water of condensation being distilled off as a prenal-water azeotrope as a vapor, at least partially condensing the vapor and separating the condensate into an aqueous phase and an organic phase and directing the organic phase partially as a reflux to the reaction column and partially discharging the organic phase as a purge stream, while continuously withdrawing an acetal fraction comprising diprenyl acetal of prenal from the reaction column, wherein the reaction temperature is below 100 °C, the catalyst is nitric acid and the concentration of the nitric acid is below 500 ppm; b) continuously subjecting the acetal fraction in a cleaving column to cleaving conditions in the presence of at least one catalyst with elimination of prenol while continuously withdrawing from the cleaving column a cleaving fraction containing at least one of prenyl (3-methyl-butadienyl) ether and 2,4,4-trimethyl-3-formyl-1,5-hexadiene, and optionally containing citral, wherein the conversion rate of diprenyl acetal of prenal in step b) is maintained at above 90% and below 100%, and the unreacted diprenyl acetal is at least partially contained in the withdrawn cleaving fraction; c) reacting the cleaving fraction in a plug-flow type reactor to obtain citral; and d) recycling part of the prenol obtained in step b) to step a). The process allows for inhibiting the formation of unwanted side-products during the production of citral.

The present invention relates to an improved process for the preparation of citral (3,7-dimethyl-octa-2,6-dienal) by which citral can be obtained in high yields. Citral is a mixture of the isomeric compounds neral and geranial. Citral is a valuable intermediate for the production of various odorants and fragrances, such as geraniol. In addition, citral has also gained importance as a starting material for the production of vitamins, especially vitamin A.

DE 198 46 056 describes a process for preparing citral by thermal cleavage of 3-methyl-2-buten-1-al-diprenyl acetal, optionally in the presence of an acid catalyst, under cleavage of 3-methyl-2-buten-1-ol (prenol) to cis/trans-prenyl-(3-methyl-butadienyl)-ether, Claisen rearrangement of said butadienyl ether to 2,4,4-trimethyl-3-formyl-1,5-hexadiene, and subsequent Cope rearrangement thereof to obtain citral. The prenol, the intermediates and the citral are continuously distilled from the reaction mixture.

WO 2008/037693 discloses a method for producing citral. Said method involves the following steps:
a) 3-Methyl-3-butene-1-ol (isoprenol) is produced from isobutylene and formaldehyde;
b) 3-methyl-2-butenal (prenal) and 3-methyl-3-butenal (isoprenal) are produced from 3-methyl-3-butene-1-ol (isoprenol) by oxidative dehydration by means of an oxygen-containing gas on a silver support catalyst;
c) additional 3-methyl-2-butenal (prenal) is produced from a mixture containing 3-methyl-3-butenal (isoprenal) by isomerization;
d) 3-methyl-2-butene-1-ol (prenol) is produced from 3-methyl-3-butene-1-ol (isoprenol) by isomerization;
e) the unsaturated acetal 3-methyl-2-butenal-diprenylacetal is produced from 3-methyl-2-butene-1-ol (prenol) and 3-methyl-2-butenal (prenal) using an acidic catalyst; and
f) citral is obtained from 3-methyl-2-butenal-diprenylacetal by cleavage and subsequently rearranging.

This complex, multi-stage process is prone to unwanted side reactions that reduce the attainable citral yield. Stated otherwise, the individual steps exhibit a less than 100% selectivity and the quantity of by-products formed may be higher than desired.

Such by-products reduce the desired selectivity of the conversion and generally must be removed from the citral product prior to subsequent use. A substantial amount of energy is required to separate by-products from citral and significant losses of citral normally are encountered. Such losses may render the use of an otherwise advantageous reaction sequence commercially unattractive.

Until now, relatively little has been known about the nature of unwanted by-products and the mechanism of their formation.

The invention therefore seeks to advise reaction conditions that effectively inhibit the formation of unwanted by-products during the production of citral, as well as to remove inevitably formed by-products without compromising the formation of citral and its building blocks.

The problem is solved by the following process and the preferred embodiments thereof.

The invention relates to a process for the preparation of 3,7-dimethyl-octa-2,6-dienal (citral) comprising the steps of:
a) continuously condensing prenol with prenal in the presence of at least one catalyst in a reaction column with water of condensation being distilled off as a prenal-water azeotrope as a vapor, at least partially condensing the vapor and separating the condensate into an aqueous phase and an organic phase and directing the organic phase partially as a reflux to the reaction column and partially discharging the organic phase as a purge stream, while continuously withdrawing an acetal fraction comprising the diprenyl acetal of prenal from the reaction column, wherein the reaction temperature is below 100 °C, the catalyst is nitric acid and the concentration of the nitric acid is below 500 ppm;
b) continuously subjecting the acetal fraction in a cleaving column to cleaving conditions in the presence of at least one catalyst with elimination of prenol while continuously withdrawing from the cleaving column a cleaving fraction containing at least one of prenyl (3-methyl-butadienyl) ether and 2,4,4-trimethyl-3-formyl-1,5-hexadiene, and optionally containing citral, wherein the conversion rate of diprenyl acetal of prenal in step b) is maintained at above 90% and below 100%, and the unreacted diprenyl acetal is at least partially contained in the withdrawn cleaving fraction;
c) reacting the cleaving fraction in a plug-flow type reactor to obtain citral; and
d) recycling part of the prenol obtained in step b) to step a).

The overall reaction sequence is illustrated by the reaction scheme below.

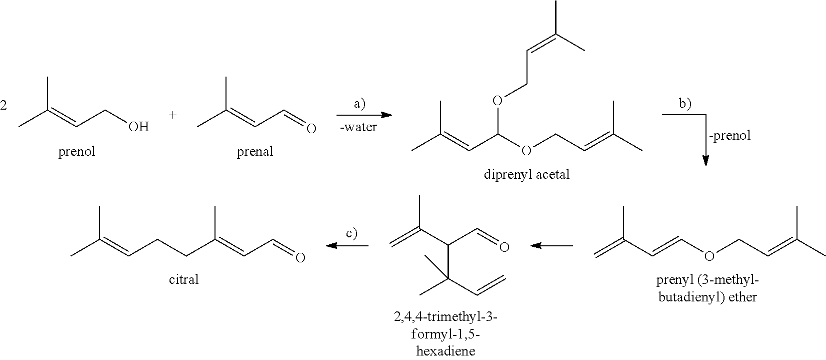

In step a), the unsaturated acetal 3-methyl-2-butenal-diprenyl acetal (herein referred to as “diprenyl acetal of prenal” or “diprenyl acetal”) is formed from prenol and prenal using a catalyst. For this purpose, prenal is reacted together with prenol in the presence of catalytic amounts of nitric acid, wherein the concentration of the nitric acid is below 500 ppm and the reaction temperature is below 100 °C, and with separation of the water formed during the reaction in a reaction column.

In step b), the resulting 3-methyl-2-butenal diprenyl acetal (diprenyl acetal) of step a) is cleaved in the presence of a catalyst in a cleaving column with elimination of 3-methyl-2-buten-1-ol (prenol) to give prenyl (3-methylbutadienyl) ether. Claisen rearrangement of the obtained prenyl (3-methylbutadienyl) ether yields 2,4,4-trimethyl-3-formyl-1,5-hexadiene which subsequently undergoes Cope rearrangement yielding 3,7-dimethyl-2,6-octadienal (citral).

In order to render the process economically viable, the prenol obtained in step b) is recycled to step a). However, it has now been found that unwanted by-products can accumulate in the recycle stream. Problems may not be apparent until large amounts of by-product accumulate in the recycle loop. The present invention, therefore, suggests to discharge a part of the organic entrainer liquid (that is the condensed distillate after removal of aqueous phase) at the top of the reaction column in which prenol is condensed with prenal, to purge the unwanted by-products. The by-products are concentrated in this organic entrainer liquid, hence the loss of valuables is minimized.

The structure of by-products that may accumulate in the recycle loop from step b) to step a) has been elucidated. The by-products were identified as by-products 1a and 1b shown below.

In a preferred embodiment, the rate of the purge stream is such that a total stationary concentration of by-products 1a and 1b

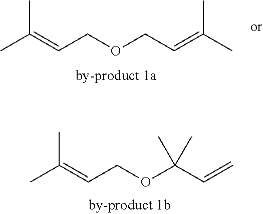

is maintained below 7 wt.-%, preferably 2 to 7 wt.-%, in the feed to step a).

“The feed to step a)” means the total of the supply of fresh prenol and prenal, and the prenol recycled from steb b).

The following by-products 2, 3, 4, 5a and 5b have been identified which are formed in step b) and c) under adverse reaction conditions:

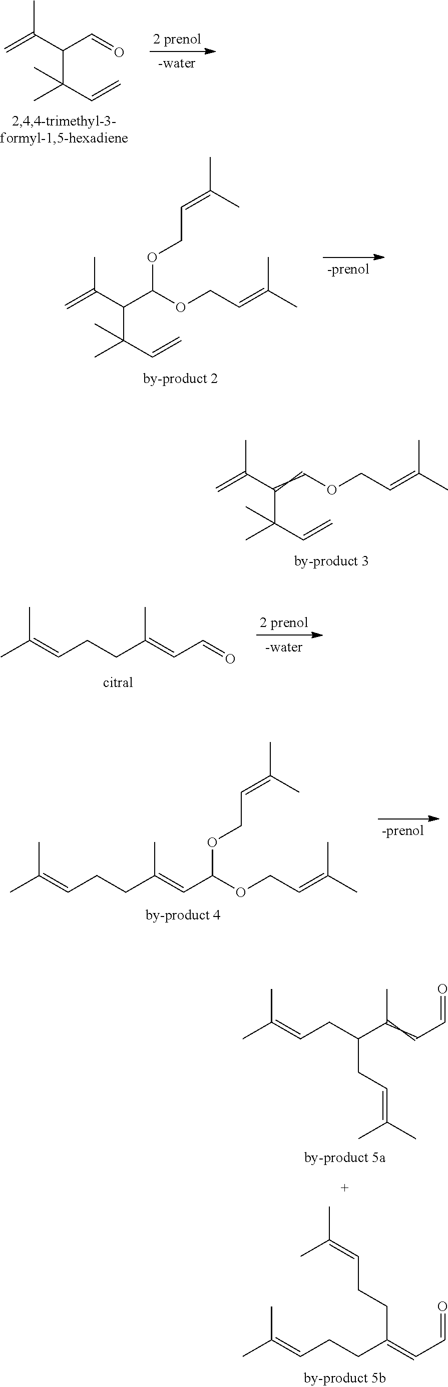

It has surprisingly been found that when the conversion rate of diprenyl acetal of prenal in step b) is driven to full conversion, the concentration of by-products increases sharply. According to the invention, the conversion rate of diprenyl acetal of prenal in step b) is maintained at above 90% and below 100%. Preferably, the conversion rate of diprenyl acetal of prenal in step b) is maintained equal to or below 99.5%, preferably equal to or below 99%, such as equal to or below 98%, or equal to or below 97.5%, or equal to or below 97%. Preferably, the conversion rate of diprenyl acetal of prenal in step b) is maintained above 91%, such as above 92%, or above or 93%, or above 94%, or above 95%. In suitable embodiments, the conversion rate of diprenyl acetal of prenal in step b) is above 94% and equal to or below 99%, such as above 95% and equal to or below 98%. Lower conversion rates will render the process economically unprofitable or will otherwise necessitate recovery and recycling of unreacted diprenyl acetal. Complete conversion in step b) is however undesirable as it results in a drop of yield of citral building blocks and increasing by-products-formation. The conversion rate is governed by various parameters including cleaving temperature, nature and concentration of the catalyst(s) in step b) and residence time in step b), i.e. in the cleaving column.

Also, it is a feature of the invention that distillation conditions in the cleaving column are effective to distill off the unreacted diprenyl acetal at least partially.

The acetal fraction is continuously subjected to cleaving conditions in a cleaving column. “Cleaving conditions” denotes reaction conditions selected such that the diprenyl acetal contained in the acetal fraction is cleaved to prenyl (3-methylbutadienyl) ether which may subsequently rearrange to 2,4,4-trimethyl-3-formyl-1,5-hexadiene and citral.

The acetal fraction comprises diprenyl acetal as a main constituent. The acetal fraction does not necessarily need to consist of pure diprenyl acetal, but may also comprise prenol, prenal and citral building blocks. In that regard, it is desirable that the diprenyl acetal content in the acetal fraction be kept above a certain level such as to avoid excessive carry-over of prenol and prenal to downstream reaction steps and subsequent separation of the latter. Further in this connection, ensuring that the diprenyl acetal content is appropriately high in the acetal fraction also means that prenol-derived by-products, such as by-product 1 exemplified below, are minimized. Accordingly, in a preferred embodiment, the acetal fraction comprises at least 65 wt.-% of diprenyl acetal, preferably at least 75 wt.-%, for example 75 to 90 wt.-%.

Rising the content of diprenyl acetal in the acetal fraction beyond a certain point reaches a point of rapidly diminishing returns. An economic balance must be taken between the improvement due to the increased content and the cost of achieving such a content. The temperature stressing induced by additional separation stages and increased residence time may even lead to decomposition of diprenyl acetal, possibly induced by traces of the acetalization catalyst(s).

Since cleavage of the diprenyl acetal may take place to a certain extent already under the conditions of the condensation reaction between prenal and prenol, attempting to drive said condensation to completion may lead to premature formation of citral building blocks which may result to undesired formation of by-product 3 and 5 (i.e. arising by reaction of said citral building blocks with prenal and prenol).

Step b) is carried out in a cleaving column. Suitably, the cleaving column is a distillation column which is equipped with an evaporator and a condenser. Suitable internals for the cleaving column are trays, packings and, in particular, structured packings made of sheet metal or metal mesh. The number of theoretical plates of the cleaving column may be in the range of from 5 to 100.

In an embodiment, the cleaving temperature in step b) is above 150 °C and below 200 °C, preferably above 155 °C and below 180 °C.

Step b) is carried out in the presence of a catalyst, preferably an acid catalyst. The catalyst can be a single catalytic species or a combination of two or more different catalytic species. Suitable acid catalysts are selected from non-volatile protic acids such as sulfuric acid, p-toluenesulfonic acid and phosphoric acid. In an embodiment, the catalyst(s) in step b) is phosphoric acid. In a preferred embodiment, the concentration of the phosphoric acid in the bottoms of the cleaving column is maintained above 100 ppm and below 1500 ppm, preferably above 200 ppm and below 1000 ppm. Higher concentrations of (acid) catalyst may result in reduced yields of citral building blocks.

Suitably, the continuous cleaving in the cleaving column of step b) may be carried out in the lower part or the sump of the distillation column acting as cleaving column. Preferably, the acetal fraction and/or the catalyst(s) are introduced into the lower part of the distillation column, into the sump of the distillation column or into the evaporator of the distillation column. If necessary, the volume of the sump of the cleaving column may be increased by a vessel in order to provide a larger reaction volume.

Typically, the bottoms from the cleaving column are a mixture of high boilers which are comprised of C5-oligomers resulting from the thermal instability of the diprenyl acetal and the citral-building blocks.

If desired, a high-boiling inert compound can be introduced into the sump of the cleaving column in order to ensure a minimum filling level of the sump and the evaporator. Suitable high-boiling inert compounds are selected from liquid compounds which are inert under the reaction conditions and have a higher boiling point than citral and diprenyl acetal. For example, the high-boiling inert compounds may be selected from hydrocarbons such as tetradecane, pentadecane, hexadecane, octadecane, eicosane; or ethers such as diethylene glycol dibutyl ether; white oils; kerosene oils; or mixtures thereof.

Part of the bottoms from the cleaving column is continuously withdrawn. This serves to avoid accumulation of high boilers. As it is a critical feature of the invention that the cleavage of the diprenyl acetal in step b) is carried out at less than complete conversion, the withdrawn part of the bottoms from the cleaving column may also comprise unreacted diprenyl acetal.

Suitably, the distillation conditions are selected such that the diprenyl acetal is predominantly retained in the lower part or the sump of the distillation column. During the cleaving reaction, a cleaving fraction is continuously withdrawn from the cleaving column, the cleaving fraction containing at least one of prenyl (3-methyl-butadienyl) ether and 2,4,4-trimethyl-3-formyl-1,5-hexadiene, and optionally containing citral. For the ease of reference, prenyl (3-methyl-butadienyl) ether, 2,4,4-trimethyl-3-formyl-1,5-hexadiene and citral are collectively referred to as “citral building blocks”. This is because the former are intermediates on the reaction route to citral and can be converted into citral in the subsequent step c).

The conversion rate of diprenyl acetal of prenal in step b) is maintained below full conversion and the unreacted diprenyl acetal is at least partially contained in the withdrawn cleaving fraction. The concentration of unreacted diprenyl acetal in the cleaving fraction may be from 1 to 6 wt.-%, relative to citral building blocks.

Additionally, the prenol formed during the cleaving reaction in step b) is continuously removed from the reaction mixture, generally at the top of the cleaving column.

The cleaving fraction together with the formed prenol may be withdrawn at the top of the distillation column.

Alternatively and preferably, it is also possible to withdraw the cleaving fraction in liquid or vaporous form at a side draw of the distillation column. The side draw preferably is located in the middle or lower part of the cleaving column, in particular 2 to 20 theoretical plates above the addition point of the acetal fraction. Preferably, the cleaving column comprises 2 to 80 theoretical plates above the side draw.

The feed rate of the acetal fraction is adjusted so as to control the residence time of the diprenyl acetal in step b). In an embodiment, the residence time in step b) is above 5 min and below 90 min, preferably above 15 min and below 80 min. Residence times in this interval represent a good trade-off between yield of reaction products and minimation of by-product formation.

In step c), the cleaving fraction is reacted in a plug-flow type reactor to obtain citral. To this end, the cleaving fraction is guided through the plug-flow type reactor at a suitable temperature of 100 to 200 °C for carrying out the rearrangement reaction(s) yielding citral.

Applicant has found that by employing a combination of a highly back-mixed cleaving column and a plug-flow reactor, it is possible to increase the selectivity and the yield of the cleaving reaction. All of the catalyst(s) required for the cleaving reaction is preferably introduced into the cleaving column in step b) and preferably, no catalyst is introduced into the plug-flow reactor.

The acetal fraction which is subjected to step b) is formed in step a) from prenol and prenal. According to the invention, in step a), prenol is continuously condensed with prenal in the presence of at least one catalyst in a reaction column while continuously withdrawing the acetal fraction of prenal from the reaction column. Further optimization of the process relates to the conditions of step a).

In step a), the formation of the following by-products has been observed under adverse reaction conditions:

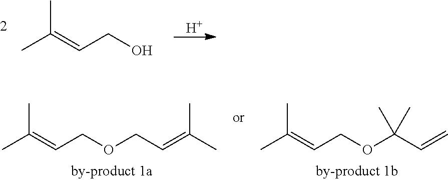

The preparation of unsaturated acetals by reacting olefinically unsaturated aliphatic compounds with allyl alcohols in a reaction column in the presence of a distillable acid is known per se. For this purpose, a mixture of at least 2 mol of prenol and 1 mol of prenal may be introduced into the reaction column, while water formed during the reaction is distilled off overhead and removed by means of a phase separator. Then, the diprenyl acetal may be removed as crude product from the bottoms or the evaporator of the reaction column. Due to the thermal instability of the diprenyl acetal, extensive purification of the crude diprenyl acetal is generally not desirable. However, concentration of the crude diprenyl acetal, e.g. in a short path evaporator, may be advantageous. Thereby, unreacted prenal and prenol are removed from the crude diprenyl acetal before the latter is directed to the cleaving column. Suitably, essentially no aldehyde is present in the crude diprenyl acetal that is directed to the cleaving column.

Suitably, the apparatus for the preparation of unsaturated acetals comprises a distillation column which is used as reaction column. The vapors rising at the top of the reaction column are condensed in a condenser and passed into a phase separation vessel, where the water separates as the lower phase. The upper phase mainly consists of organic compounds such as unreacted aldehyde, i.e. prenal, unreacted alcohol, i.e. prenol, and low-boiling secondary compounds, e.g. formates of prenol. Most of the organic phase is recycled to the top of the reaction column as a reflux, and a smaller portion is discharged to remove by-components.

Suitably, the amount of reflux per 1000 kg of freshly added aldehyde is in the range of from 200 kg to 50000 kg, preferably 1000 kg to 20000 kg. Depending on the purity of the feed, the amount of the discharged portion per 1000 kg of freshly added aldehyde is in the range of from 1 kg to 400 kg, preferably 5 kg to 200 kg.

The reaction temperature in step a) is below 100 °C, preferably in the range of from 70 to 80 °C. In an embodiment, the reaction pressure in step a) is below 150 mbar, preferably in the range of from 90 to 110 mbar. Suitably, the residence time of the reaction mixture in the reactor is in the range of from 0.1 s to 10 h, preferably 60 s to 2 h.

According to the invention, step a) is carried out in the presence of a catalyst, wherein the catalyst is nitric acid. The concentration of the nitric acid is below 500 ppm, more preferably in the range of from 100 to 300 ppm, relative to the total amount of the starting materials prenol and prenal. Lower amounts of (acid) catalyst may result in a low conversion in the reaction column. Higher amounts of (acid) catalyst may disadvantageously result in increased formation of by-products and in decreased selectivities.

Suitably, the catalyst is added to the reaction column, preferably to the lower part of the reaction column. In a preferred embodiment, it is added to the evaporator. It is also possible to add the nitric acid at different locations, e.g. at two or more points in the reaction column.

The point of addition of fresh aldehyde and/or fresh alcohol is not critical. The aldehyde and alcohol may be added separately at different points in the reaction column. Preferably, aldehyde and alcohol are combined with the effluent from the condenser. The amount of freshly added alcohol is controlled such that the ratio of alcohol to aldehyde is in the range of from 1 to 3, preferably 1.5 to 2.5.

In step d), prenol eliminated in step b) is recycled to step a). This allows for improved yields to be achieved in the process of the invention.

Applicants have found that it is important to control the concentration of contaminants contained of the prenol which is recycled to step a). Specifically, the concentration of 2,4,4-trimethyl-3-formyl-1,5-hexadiene of the prenol recycled from step b) into step a) is preferably controlled such that the concentration of 2,4,4-trimethyl-3-formyl-1,5-hexadiene in step a) is below 1 wt.-%, preferably below 0.5 wt.-%, relative to the total weight of prenol and prenal. Similarly, the concentration of citral of the prenol recycled from step b) into step a) is preferably controlled such that the concentration of citral in step a) is below 1 wt.-%, preferably below 0.1 wt.-%, relative to the total weight of prenol and prenal.

It has been found by the inventors that it is advantageous to control the amount of 2,4,4-trimethyl-3-formyl-1,5-hexadiene and/or citral which is introduced into step a) together with the recycled prenol from step b). Higher concentrations of 2,4,4-trimethyl-3-formyl-1,5-hexadiene and/or citral as described above result in the formation of by-products.

Prenol useful as a starting material for the invention may be obtained by reacting at least one formaldehyde source and isobutylene to obtain 3-methylbut-3-en-1-ol (isoprenol) in a reactor, in general at elevated temperature and pressure, and subjecting the obtained isoprenol to isomerization.

In one embodiment, the isoprenol is obtained by mixing and injecting at least one formaldehyde source and isobutylene into a reactor through at least one nozzle and reacting the at least one formaldehyde source and isobutylene under supercritical conditions. In order to achieve supercritical conditions, formaldehyde and isobutylene are preferably reacted at a temperature of at least 220 °C, for example in the range of 220 to 290 °C, and an absolute pressure of at least 200 bara. The reaction of isobutene and formaldehyde may be carried out in the presence of at least one catalyst such as an amine base, e.g. hexamethylenetetramine (urotropin).

In one embodiment, the isoprenol is obtained by mixing and injecting the at least one formaldehyde source and isobutylene into a reactor through a plurality of nozzles operated in parallel and reacting the formaldehyde source and isobutylene under supercritical conditions; wherein the reactor comprises a vertically disposed vessel, a sidewall, an upper portion and a lower portion; and wherein the formaldehyde source and isobutylene are injected into a mixing chamber of the reactor disposed in the upper portion and a fluid comprising formaldehyde and/or isobutylene and/or isoprenol is passed from the mixing chamber into a post-reaction chamber disposed in the lower portion; and providing draft tubes arranged essentially concentrically underneath each of the nozzles in the mixing chamber, the draft tubes providing downcomer conduits within the draft tubes and a riser conduit outside of the draft tubes, so that the formaldehyde source and isobutylene injected through the nozzles travel generally downward in the downcomer conduits, a fluid comprising formaldehyde and/or isobutylene and/or isoprenol is then diverted in a generally upward direction in the riser conduit, and the fluid is back-mixed with the injected formaldehyde source and isobutylene.

Formaldehyde may be provided as a liquid, for example as a solution of paraformaldehyde. Preferably, the at least one formaldehyde source comprises or is an aqueous formaldehyde solution.

While initial rapid and intense mixing of reactants is desirable, it may be advantageous to continue and complete the reaction under conditions of limited back-mixing. Thus, the reaction mixture may be passed into a post-reaction chamber disposed after the reactor or in a lower portion of the reactor. In the post-reaction chamber, back-mixing is limited.

In one embodiment, the reactor comprises an upper portion and a lower portion. Injecting and mixing of the reactants occurs in a mixing chamber of the reactor disposed in the upper portion, and a fluid comprising formaldehyde and/or isobutylene and/or isoprenol is passed from the mixing chamber into a post-reaction chamber disposed in the lower portion.

Further details regarding reacting at least one formaldehyde source and isobutylene to obtain isoprenol may be found in WO 2020/049111 A1.

In one embodiment, reacting at least one formaldehyde source and isobutylene comprises mixing and injecting the at least one formaldehyde source and isobutylene into an internal loop reactor through at least one nozzle into first conduit(s), the internal loop reactor comprising:
a vertically disposed cylindrical vessel comprising a sidewall;
at least one draft tube having a tube inlet end and a tube outlet end, arranged vertically within the vessel, the draft tube(s) being arranged concentrically to the nozzle(s), and having an inner surface and an outer surface, wherein the draft tube(s) provide(s) the first conduit(s) within the draft tube(s), and a second conduit outside of the draft tube(s) and within the sidewall, the first conduit(s) being in fluid communication with the second conduit;
reactor fluid outlet means;
   wherein the inner surface of the draft tube(s) convexly curves so that the first conduit(s) exhibit(s) an annular constriction of the cross-section between the tube inlet end and the tube outlet end; wherein the constriction is located closer to the tube inlet end; wherein the convex curvature of the inner surface of the draft tube(s) extends over at least 70%, preferably at least 80%, most preferably at least 90% of the length of the draft tube; and
   wherein the outer surface of the draft tube(s) convexly curves so that the draft tube(s) exhibit(s) a circumferential protuberance between the tube inlet end and the tube outlet end, which circumferential protuberance is preferably located closer to the tube outlet end; wherein the convex curvature of the outer surface of the draft tube(s) extends over at least 70%, preferably at least 80%, most preferably at least 90% of the length of the draft tube; and
   wherein the edges of the draft tube(s) are rounded so that the at least one formaldehyde source and isobutylene injected through the nozzles travel generally downward in the first conduit(s) to obtain a reacted fluid, the reacted fluid is then diverted in the opposite direction so as to travel through the second conduit and is subsequently back-mixed with the injected fluid.

This configuration of the draft tube(s) allows control of the boundary layer flowing over the edge of the draft tube. When the angle of attack of a flow with respect to a solid body reaches a certain limit, the adverse pressure gradient becomes too large for the flow to negotiate it. At this point, the flow separates from the upper surface of the body, resulting in a condition commonly known as stall. The configuration allows for decreased flow separation or a delay in flow separation, respectively. Decreased flow separation allows for reduced liquid friction and thus leads to a lower pressure drop along the streamline of the recirculating flow, which in turn results in a higher circulation ratio of the configuration. The curved shape of the inner surface of the draft tube wall guides the fluid through the draft tube in an optimized manner, comparable to the flow of fluid over an airfoil.

The inner surface of the draft tube curves in the longitudinal direction of the draft tube, or in other words has a convex shape, so that the first conduit exhibits a minimum cross-section between the tube inlet end and the tube outlet end. This means that the cross-section of the first conduit decreases from the cross-section at the tube inlet end to a minimum cross-section and increases from the minimum cross-section to the cross-section at the tube outlet end.

The draft tube has a curved, approximately conical section between the tube inlet end and the constriction, being wide at the inlet end and narrower at the constriction. At least some of the fluid flowing downstream through the draft tube is deflected so as to flow along the inner surface of the draft tube until the draft tube ends. The flow through the tube predominantly remains attached, thus generating less pressure loss. In the vicinity of the constriction, the fluid flowing downstream through the draft tube is accelerated. Between the constriction and the tube outlet end, the cross-sectional area of the draft tube widens again. Consequently, the area variation, in conjunction with mass conservation, will force the velocity through the larger area to be slower than through the smaller one, accompanied by a conversion of the dynamic pressure into static pressure. Acceleration of the fluid flowing downstream through the draft tube in the vicinity of the constriction adds a radial velocity component to the flow, increasing the mixing between circulating flow and injected flow. By avoiding flow separation in this case, no significant pressure loss occurs.

In a preferred embodiment, the nozzles are two-component nozzles. It is especially preferable that a two-component nozzle is designed so as to provide an annular jet of isobutylene around a central jet of the at least one formaldehyde source, and that the injection velocities of these two jets are different. In this embodiment, the jet of isobutylene has a large shear surface towards both the central jet of the at least one formaldehyde source and the reaction mixture in the reactor, allowing for favorable fast mixing of the reactants.

In a preferred embodiment, the loop reactor comprises deflector means arranged between the nozzle and the draft tube, the deflector means being suitable for deflecting fluid travelling in the second conduit in the opposite direction.

The deflector means suitably comprise a surface which is concave relative to the end of the draft tube which defines the tube inlet end. In a preferred embodiment, the deflector means have a partial toroidal surface. It is especially preferred that the deflector means are provided in the shape of the upper portion of a ring torus bisected in a plane parallel to the toroidal direction. This shape allows for an especially efficient deflection of the fluid travelling in the second conduit. The deflector means may allow for a stabilization of the injected fluid stream. This is especially relevant when the flow rate of the fluid travelling in the second conduit is not uniform across the cross section of the reactor, which may lead to an eccentricity of the injected fluid stream. Such an eccentricity may cause a decrease in circulation ratio if left unattended.

When the first conduit is downcomer conduit and the second conduit is a riser conduit, it is preferred that the shape of the deflector means constitutes the upper portion of a ring torus bisected in a plane parallel to the toroidal direction, wherein the ring torus is bisected at at least 50% of its height, such as at least 55% or 65% of its height. Thus, the upper portion of the ring torus is the same size or smaller than the lower portion of the ring torus. In another preferred embodiment, the shape of the deflector means constitutes the upper portion of a ring torus bisected in a plane parallel to the toroidal direction wherein the ring torus is bisected at at most 85% of its height, for example 80% of its height. In these ranges, the entry of the deflector means is angled especially suitable for fluid deflection.

High temperatures are required to obtain a high isoprenol yield in the reaction of formaldehyde with isobutylene. Effective removal of the heat is critical for the product quality and process safety. The heat removed from the isoprenol is used for raising the temperature of isobutylene before it enters the reactor. The stream of the hot isoprenol contains sensible heat from the chemical reaction. The sensible heat is potentially reclaimable energy that can be reused.

Advantageously, reacting at least one formaldehyde source and isobutylene preferably comprises heat-exchanging a stream of hot isoprenol withdrawn from the reactor with a isobutylene stream directed to the reactor; wherein heat-exchanging is performed in at least two shell-and-tube heat exchangers; each of the heat exchangers comprising a plurality of tubes and a shell-side heat exchange passage; wherein the hot isoprenol is directed through the tubes of the heat exchangers; and the isobutylene is guided through the shell-side passage, and at least two of the heat exchangers are connected in series with regard to both the shell-side flow and the tube-side flow.

Such configurations allow for prolonging operation intervals between maintenance disruptions in such a method. The term “maintenance disruptions” is intended to mean a shutdown of the process that becomes necessary at recurring intervals in order to clear the tubes of the heat exchanger that have been clogged by fouling. An indicator of a necessity of a maintenance disruption is typically when isobutylene leaving the last heat exchanger is insufficiently pre-heated and that even a subsequent heater is hardly able to put in additional external heat into the isobutylene to bring isobutylene to the required temperature before it enters the reactor. In the present configuration, the pre-heating of the isobutylene stream can be maintained for a longer time at levels high enough so that the desired temperature of the isobutylene can easily be reached before the isobutylene enters the reactor.

One particular area prone to fouling in conventional shell-and-tube heat exchangers is the tube area near the tube sheet near the inlet where the tube-side fluid leaves the individual tubes. Excessive fouling in this area can cause clogging of individual tubes and fluid stagnation along the entire length of these tubes. The fluid stagnation generally leads to reduced heat-transfer performance.

As a further consequence of the decreased heat transfer performance caused by fouling, the energy required in a heater to adjust the temperature of the pre-heated isobutylene stream to the desired reaction temperature increases. Consequently, more additional external heat becomes necessary which is detrimental in terms of energy demand and process economy, and often has a negative impact on the carbon dioxide footprint of the product.

By using two or more heat exchangers, the impact of fouling in individual tubes on the overall heat exchange capacity is reduced in comparison to arrangements where only a single heat exchanger is used. As a consequence, the heat transfer rates are maintained at a desired level for longer periods, hence prolonging operation intervals between maintenance disruptions, and the pre-heating of the isobutylene stream requires less additional external heat compared to a plant with a single heat exchanger in an advanced state of fouling.

Separation of the isoprenol from unreacted formaldehyde is not a trivial task. This difficulty arises from the fact that monomeric formaldehyde (as well as polymeric formaldehyde) forms both hydrates with water and hemiformals with isoprenol. The hydrates and hemiformals of varying formaldehyde polymerization degree have intermingling boiling points.

It has, however, been found that formaldehyde can be separated virtually completely from isoprenol via distillation at a temperature at which the hemiformal is cleaved to formaldehyde and isoprenol, so that the formaldehyde can be easily separated from the isoprenol.

Hence, crude isoprenol may be purified by subjecting a stream of crude isoprenol containing isoprenol, water and formaldehyde, or an isoprenol containing fraction thereof, to distillation in a low-boiler separation tower operated at a pressure of 2 bara or higher, preferably 2.5 bara or higher, to obtain a distillate stream containing aqueous formaldehyde and a bottoms stream containing isoprenol essentially free of formaldehyde.

In particular, it has been found that the formaldehyde can be separated virtually completely from isoprenol and concentrated aqueous formaldehyde suitable for recycling into the isoprenol synthesis can be obtained in a distillation train involving a first distillation at a temperature at which the equilibrium is shifted towards the hemiformal of formaldehyde and isoprenol, so that essentially all formaldehyde remains in the bottoms of the distillation, and a second distillation at a temperature at which the hemiformal is cleaved to formaldehyde and isoprenol, so that the formaldehyde can be easily separated from the isoprenol.

In order to permit a first distillation at a temperature below the isoprenol-formaldehyde dissociation temperature and a second distillation at a temperature above the isoprenol-formaldehyde dissociation temperature, two low-boiler separation towers operated at different pressures are envisioned. Hence, at the relatively low pressure prevailing in the first low-boiler separation tower, a first distillate containing water and low-boilers essentially free of formaldehyde is obtained. At the relatively high pressure prevailing in the second low-boiler separation tower, a virtually all formaldehyde is separated from the isoprenol. This process thus allows for obtaining isoprenol essentially free of formaldehyde.

Hence, in a more preferred embodiment, the purification process comprises
(i) directing the stream of crude isoprenol to a first low-boiler separation tower operated at a pressure of 1.5 bara or lower, to obtain a first bottoms stream containing isoprenol and formaldehyde, and a first distillate stream containing water and low-boilers;
(ii) directing the first bottoms stream to a second low-boiler separation tower operated at a pressure of 2 bara or higher, to obtain a second distillate stream containing aqueous formaldehyde, and a second bottoms stream containing isoprenol; and
(iii) directing the second bottoms stream to a finishing tower to obtain pure isoprenol as a distillate stream, and a bottoms stream containing high-boilers.

The second distillate stream constitutes concentrated aqueous formaldehyde fit for recycle into the isoprenol synthesis.

Suitably, the second low-boiler separation tower is operated at a pressure of 2.5 bara or higher, preferably 2.8 bara or higher, most preferably 2.9 bara or higher. The bottoms temperature of the second low-boiler separation tower is preferably in the range of 160 to 200 °C, more preferably 170 to 185 °C, most preferably 175 to 180 °C. The temperature at the top of the second low-boiler separation tower is preferably in the range of 115 to 160 °C, more preferably 125 to 145 °C.

In a particularly preferred embodiment, the second low-boiler separation tower is operated at a pressure in the range of 2.9 to 3.5 bara, a bottoms temperature in the range of 175 to 180 °C and a temperature at the top in the range of 130 to 140 °C.

Further information on the process for recovering isoprenol essentially free of formaldehyde may be found in WO 2022/189652 A1.

The obtained isoprenol may be subjected to catalytic isomerization by bringing a reactant stream comprising isoprenol into contact with at least one heterogeneous isomerization catalyst, so as to obtain prenol.

Isomerization of isoprenol to 3-methyl-2-buten-1-ol (prenol) may be carried out over a supported noble metal, preferably in the presence of hydrogen. A preferred catalyst is a fixed bed catalyst containing palladium and selenium or tellurium or a mixture of selenium and tellurium supported on silicium dioxide. The isomerization is carried out at a temperature of 50 to 150 °C to produce a reaction mixture of prenol and isoprenol. The isoprenol can be recycled. Further details are provided in WO 2008/037693.

When isoprenol is subjected to catalytic isomerization, it may be favorable to maintain in the reactant stream a weight ratio of formaldehyde to isoprenol of less than 0.04, preferably less than 0.03, in particular less than 0.02, or less than 0.01. In still more preferred embodiments, the weight ratio of formaldehyde to isoprenol is maintained at less than 0.002, or less than 0.001.

It has been found that the presence of formaldehyde in the reactant stream is detrimental to the activity and selectivity of the process and may accelerate catalyst deactivation and/or poisoning.

Hydrogen is required for isomerizing isoprenol to prenol. It is believed that the poisoning mechanism resulting from the presence of formaldehyde involves the dehydrogenation of formaldehyde to carbon monoxide, which is more strongly chemisorbed on the catalyst than hydrogen.

A further cause of catalyst deactivation, which may occur in combination with the previously mentioned cause of catalyst poisoning, is the formation of paraformaldehyde or trioxane which may deposit, in the form of solids, on the catalyst and shield the catalytically-active surfaces from the isoprenol being processed. This leads to progressive deactivation of the catalyst.

It is expected that further side products typically comprised in isoprenol due to the manufacturing process, such as prenal or isoamyl alcohol, have little to no impact on the performance of the isomerization reaction.

The weight ratio of formaldehyde to isoprenol in the reactant stream may be maintained at a certain level or less. Reducing the weight ratio of formaldehyde to isoprenol in the reactant stream beyond a certain point, however, reaches a point of rapidly diminishing return. Formaldehyde removal involves additional equipment and operating costs. An economic balance must be taken between the improvement due to reducing the ratio and the cost of maintaining such a ratio. Hence, the weight ratio of formaldehyde to isoprenol is preferably not lower than 0.0005 or, in some instances, not lower than 0.005.

The presence of formaldehyde in the reactant stream is due to two main sources. Formaldehyde may be contained in the fresh feed stream sent to the reactor, that is as an impurity originating from the isoprenol manufacture step. All the formaldehyde that cannot be separated in the purification step following the isoprenol synthesis ends up in the reactant stream.

In addition, formaldehyde is also generated in situ. Part of the isoprenol splits back to isobutene and formaldehyde.

Since the double bond isomerization of isoprenol is an equilibrium reaction, conversion is necessarily incomplete. For economic operation of the process, the unconverted isoprenol has to be removed and recycled. Recycling of isoprenol may inadvertently (re) introduce formaldehyde into the isomerization step if no steps to purify the stream containing unreacted isoprenol are taken.

Reducing the weight ratio of formaldehyde to isoprenol in the reactant stream can be accomplished in several different ways. In an embodiment, formaldehyde is removed from the unreacted isoprenol stream prior to combining the unreacted isoprenol stream with the fresh feed stream.

In an embodiment, the unreacted isoprenol stream is combined with the fresh feed stream and formaldehyde is removed from the combined stream.

Alternatively, it is feasible to mix the unreacted isoprenol stream with an amount of a sufficiently purified fresh feed stream so as to give in the combined stream a desired weight ratio of formaldehyde to isoprenol.

Formaldehyde may be removed from isoprenol streams by a conventional separating method such as distillation, selective adsorption and or selective reaction, in particular by the purification process involving the pressure-swing distillation as described above.

Isoprenol, in particular the isoprenol obtained as described above, may be converted to prenal, involving isomerization and an oxidative dehydrogenation in any order. Thus, it is possible to first isomerize isoprenol to prenol, and subsequently oxidize prenol to prenal; or, to first oxidatively dehydrogenate isoprenol to isoprenal, and optionally isomerize at least part of the isoprenal to prenal.

Prenol, in particular the prenol obtained as described above, may be oxidized so as to obtain prenal by bringing a reactant stream comprising prenol into contact with at least one oxidant and at least one oxidation catalyst, preferably in the presence of a liquid phase.

Suitable oxidants include hydrogen peroxide and oxygen, in particular oxygen.

The oxidation is preferably carried out in the presence of a liquid phase and with oxygen as the oxidant. The liquid phase preferably comprises at least 25 wt.-% of water, more preferably at least 50 wt.-% of water or at least 70 wt.-% of water, based on the total weight of the liquid phase, determined at a temperature of 20 °C and a pressure of 1 bar. It has been found that these conditions allow for a simple and efficient process for preparing prenal from prenol.

The oxidation is typically carried out in the presence of at least one oxidation catalyst selected from the group consisting of platinum, palladium and gold. Preferably, the at least one oxidation catalyst comprises platinum. In a preferred embodiment, the at least one oxidation catalyst is a supported catalyst.

The oxidation is suitably carried out at a temperature of 20 °C to 100 °C, preferably at a temperature of 20 °C to 70 °C. The oxidation is suitably carried out under a partial pressure of oxygen between 0.2 and 8 bar.

Oxidative dehydrogenation of isoprenol typically comprises bringing a reactant stream, in particular a gaseous reactant stream, comprising isoprenol into contact with at least one heterogeneous oxidative dehydrogenation catalyst, in particular at least one silver-containing heterogeneous oxidative dehydrogenation catalyst, in the presence of molecular oxygen. The at least one heterogeneous catalyst may consist of an inert support having a smooth surface having an active layer of silver. Alternatively, massive (full-metal) silver bodies may be used.

In an embodiment, oxidative dehydrogenation is carried out by passing the isoprenol through a plurality of reaction tubes of a shell-and-tube heat exchange reactor comprising
a shell-side heat exchange passage for circulating a heat transfer medium and a reaction passage comprising the plurality of reaction tubes;
an inlet for introducing the reactant stream to the reaction passage; and
an outlet from the reaction passage for recovering an effluent stream from the reaction tubes;
   wherein the reaction tubes comprise
   a reactant pre-heating zone adjacent to the inlet, and
   a reaction zone downstream of the reactant pre-heating zone, the reaction zone having a catalytically active wire matrix insert having silver at least on a part of its surface.

The term “reactant pre-heating zone” denotes a section of the reaction tube, i.e. a section inside the reaction tube, where essentially no catalytic oxidative dehydrogenation reaction occurs and where the gaseous stream through the reaction tubes is heat-exchanged via the tube wall with the circulating heat transfer medium. The pre-heating zone upstream of the reaction zone involves net heat flow into the reaction tube and ensures that the reactant stream is sufficiently heated up to a temperature close to or at the reaction temperature when it reaches the reaction zone.

Upon contact with the catalytic surface, the oxidative dehydrogenation reaction immediately starts. Otherwise, in the event when a “cold” reactant stream reaches the catalytic surface such that the reaction onset temperature of the reaction is not reached, coke formation may occur. Less coke formation advantageously leads to a prolonged reactor operation without the necessity of burning off the coke from the catalytic surface.

Preferably, the reactant pre-heating zone is adapted to allow for laminar flow of the reactant inside the reactant pre-heating zone. This means, the reactant pre-heating zone is devoid of any obstacles to the reactant flow that triggers a laminar-to-turbulent flow transition. Hence, the reactant pre-heating zone preferably has an essentially free cross section, i.e. the pre-heating zone is empty.

In the case of an “essentially free cross section”, the reactant pre-heating zone may be empty. Alternatively, the reactant pre-heating zone may accommodate fixtures made of a material having zero or limited catalytic activity, which fixtures have a negligible cross-section in a plane perpendicular to the longitudinal axis of the reaction tube. Said fixtures may be attached to the catalytically active wire matrix which is present in the reaction zone and allow to easily place said wire-matrix insert into or remove the same from the reaction zone. For example, the negligible mounting may be a stainless steel wire or rod.

This setup allows for heating up only the portion of the entire reactant stream that travels near the hot reaction tube wall. Consequently, the portion of the reactant stream flowing in the center of the reaction tube is not heated to the reaction temperature and blind reactions of the unstable starting materials are thus reduced or even avoided. A “blind reaction” is an unselective oxidative reaction that occurs in the absence of the catalyst. Once the reactant stream reaches the reaction zone, the oxidative deyhdrogenation reaction is initiated. Due to the exothermic nature of this reaction, energy is released and the remainder of the reactant stream is rapidly heated to the reaction onset temperature, and the reaction proceeds. This fast heat up of the predominant part of the reaction mixture reduces unwanted side-reactions and thus leads to an increased selectivity.

Alternatively, the reactant pre-heating zone may have a wire matrix insert having zero or limited catalytic activity. The wire matrix insert may reduce or eliminate temperature gradients without creating any obstruction to flow that would promote turbulent flow characteristics. A wire matrix insert is considered as having zero catalytic activity (or in other words, as being “inert”) if it does not catalyze the gas-phase partial oxidation reaction in question to a significant degree, and the chemical composition of a stream passing the wire matrix insert does not change significantly. Similarly, a matrix insert is considered as having limited catalytic activity if its catalytic activity is less than the activity of a reaction zone. In an embodiment, the wire matrix insert having zero or limited catalytic activity is made of an inert material, preferably stainless steel.

Herein, the term “reaction zone” denotes a region of the reaction tube where the catalytic gas-phase partial oxidation reaction occurs. The reaction zone comprises a catalytically active wire matrix insert having at least on a part of its surface a catalytically active precious metal. Due to the more open structure of the wire matrix contained in the reaction zone as compared to a packing of individual elements, a larger proportion of the reaction heat is discharged to the reaction tube wall by radiation and does not have to be dissipated by the reactant stream. Due the unique flow characteristic of the reactant stream through the reaction tube with the wire matrix insert in place, heat transfer via the tube wall is improved. Formation of prominent hotspots can be avoided. This in turn, avoids deposition of organic constituents of the reactant stream on the surface of the active catalyst material with concomitant pressure drop. Overall, less regular maintenance in the form of regeneration and/or replacement of the catalyst is required. The number of annual operating hours can be increased and the existing production capacities can be fully utilized, reducing operation cost and increasing profit.

In contrast to individually present catalyst bodies, the wire matrix inserts can be formed contiguously, or in one piece. Hence, placing the wire matrix inserts in the catalyst containment region of the reaction tubes, and removal therefrom is much facilitated.

The “reaction zone” may be comprised of a single contiguous reaction zone. Alternatively, the reaction zone may comprise an alternating series of regions having catalytically active wire matrix inserts and regions having an essentially free cross section or having wire matrix inserts having zero or limited catalytic activity.

A “wire matrix insert” is understood to be a self-supporting skeletal-like structure made of coiled, bent or crimped metal wire which is adapted to be inserted into a reaction tube of a shell-and-tube reactor. The wire matrix insert has a more voluminous structure than a longitudinal wire.

A fixture such as a stainless steel wire or rod may be attached to the wire matrix insert which allows for easily placing the wire-matrix insert into or removing the same from the reaction zone.

In an embodiment, the catalytically active wire matrix inserts comprise an elongated core having a plurality of wire loops extending from the elongated core, wherein the wire loops are longitudinally arranged and helically shifted, that is, neighboring wire loops have an angular offset. The loops may be formed by helically bending the wire over the length of the wire matrix insert. In view of the ease of manufacture, the elongated core preferably comprises at least two longitudinal core wire members, which are twisted around each other to form core wire windings, and the wire loops are accommodated in the core wire windings.

The wire loops may be formed from one wire, or more than one intertwined wires, preferably 4 intertwined wires.

The wire matrix insert comprised in the reaction zone has silver at least on a part of its surface a catalytically active precious metal. The wire constituting the wire loops may be a massive silver wire, or a wire coated with silver. The core wire may be made of brass alloys, or high-grade steels. The coating layer of silver superimposed on the surface of the core has a thickness of, e.g., 10 μm. In general however, a massive silver wire has better service life and is preferred. If the wire loops are formed from more than one intertwined wires, at least one of the intertwined wires is made of a massive silver wire, or a wire coated with silver while the other intertwined wires can be made of an inert material.

A silver wire which is of the same composition throughout its cross section and comprises at least 92.5 wt.-% Ag can suitably be used. The silver wire is helically bent to form wire loops, and combined with at least two longitudinal core wire members, which are twisted around each other to form core wire windings, and the wire loops are accommodated in the core wire windings. The longitudinal core wire members can also be silver wire or inert metal wire.

In a preferred embodiment, the catalytically active wire matrix inserts comprise an elongated core having a plurality of wire loops extending from the elongated core, wherein the wire loops are longitudinally arranged and helically shifted, and the wire loops comprise a massive silver wire.

Generally, the catalytically active wire matrix inserts have a cylindrical enveloping surface with a diameter matching with the inner diameter of the reaction tubes. This includes a situation where the diameter of the cylindrical enveloping surface of the undeployed wire matrix insert is slightly larger than the inner diameter of the reaction tubes. Due to the springy or elastic nature of the wire matrix insert, it can be inserted into the reaction tubes with a slight counter pressure such that the wire loops fit tightly against the inner walls of the reaction tube.

Suitable structures of wire matrix inserts are known as such, see, e.g., GB 2 097910 Some inserts of this type are disclosed in GB patent 1 570 530. Other inserts, as well as processes for their production are disclosed in GB 2 097 910 A. Matrix inserts are commercially available from the company Cal Gavin Ltd., England, and sold under the trade name HiTRAN®.

When isoprenol is subjected to oxidative dehydrogenation, it may be favorable to maintain in the reactant stream a weight ratio of formaldehyde to isoprenol of less than 0.04, preferably less than 0.03, in particular less than 0.02, or less than 0.01. In still more preferred embodiments, the weight ratio of formaldehyde to isoprenol is maintained at less than 0.002, or less than 0.001.

The weight ratio of formaldehyde to isoprenol in the reactant stream may be maintained at a certain level or less. Reducing the weight ratio of formaldehyde to isoprenol in the reactant stream beyond a certain point, however, reaches a point of rapidly diminishing return. Formaldehyde removal involves additional equipment and operating costs. An economic balance must be taken between the improvement due to reducing the ratio and the cost of maintaining such a ratio. Hence, the weight ratio of formaldehyde to isoprenol is preferably not lower than 0.0005 or, in some instances, not lower than 0.005.

It has been found that reactor clogging and pressure drop increase are significantly affected by the presence of formaldehyde in the reactant stream. Catalyst-fouling reactions of condensation and polymerization are believed to be the principal reactions involved in carbon or coke formation on the catalyst. It is thought that this carbon formation involves thermal condensation of formaldehyde or of formaldehyde with the olefinic hydrocarbons isoprenol and (iso) prenal. In the presence of the catalyst, the primary condensation products tend to undergo dehydrogenation and polymerization type reactions and to settle on the catalyst and undergo further dehydrogenation and decomposition until carbonaceous deposits are formed.

The presence of formaldehyde in the reactant stream is due to two main sources. Formaldehyde may be contained in the fresh feed stream sent to the reactor, that is as an impurity originating from the isoprenol manufacture step. All the formaldehyde that cannot be separated in the purification step following the isoprenol synthesis ends up in the reactant stream.

In addition, formaldehyde is also generated in situ. Part of the isoprenol splits back to isobutene and formaldehyde. Since most continuous industrial processes operate at single-pass conversion levels of 50 to 60% and with recycling of the unconverted isoprenol, formaldehyde may be present in the recycling stream of unconverted isoprenol, if no steps to purify the stream containing unreacted isoprenol are taken.

The recycle stream of unconverted isoprenol has now been found to constitute the biggest source of formaldehyde contamination in the reactant stream. The process is generally carried out at partial conversions, for example at conversions of 30 to 70%, preferably 50 to 60%. An unreacted isoprenol stream is separated from the product stream. The unreacted isoprenol stream is recycled, that is, combined with a fresh feed stream comprising isoprenol to provide the reactant stream. The unreacted isoprenol stream comprises isoprenol as a main constituent, but may also comprise prenal, isoprenal, isoamylalcohol, isovaleraldehyde, isovaleric acid, prenol, formaldehyde. It can also contain traces of other C₃ and C₂ aldehydes and acids.

Reducing the weight ratio of formaldehyde to isoprenol in the reactant stream can be accomplished in several different ways. In an embodiment, formaldehyde is removed from the unreacted isoprenol stream prior to combining the unreacted isoprenol stream with the fresh feed stream.

In an embodiment, the unreacted isoprenol stream is combined with the fresh feed stream and formaldehyde is removed from the combined stream.

Alternatively, it is feasible to mix the unreacted isoprenol stream with an amount of a sufficiently purified fresh feed stream so as to give in the combined stream a desired weight ratio of formaldehyde to isoprenol.

Formaldehyde may be removed from isoprenol streams by a conventional separating method such as distillation, selective adsorption and or selective reaction, in particular by the purification process involving the pressure-swing distillation as described above.

Prior to contacting with the at least one oxidative dehydrogenation catalyst or with the at least one oxidation catalyst, respectively, the (iso)prenol may advantageously be treated to remove organically bound nitrogen from the (iso)prenol by contacting the (iso)prenol with a weakly acidic solid adsorbent. In other words, the (iso)prenol may be depleted of organically bound nitrogen by this process.

The term “organically bound nitrogen” is intended to denote any compound containing at least one nitrogen atom directly bound to one or more carbon atoms. For example, such compounds containing at least one nitrogen atom may be selected from amines, such as ethylamine, trimethylamine, aniline, pyridine or piperidine. An amine particularly significant in practice is hexamethylenetetramine (urotropin). (Iso)prenol may comprise about 5 to 30 ppm of organically bound nitrogen.

The weakly acidic solid adsorbents have been found to be capable of adsorbing organically bound nitrogen in the presence of abundant (iso)prenol while not interfering with the reactive carbon-carbon double bond.

The weakly acidic adsorbent may include an adsorbent material having sufficient acidity to adsorb the organically bound nitrogen from the (iso)prenol. In an embodiment, the solid adsorbent is a crosslinked resin having phosphonic functional groups. Preferably, the resin polymer is a vinyl aromatic copolymer, preferably crosslinked polystyrene and more preferably a polystyrene divinylbenzene copolymer. Other polymers having a phosphonic functional group may also be used. Preferably, the crosslinked resin having phosphonic functional groups is of the macroporous type. A preferred solid adsorbent is Purolite S956.

The resin is typically used in bead form and loaded into a column. The (iso)prenol is passed through the column, contacting the resin beads. During contact, the organically bound nitrogen in the (iso)prenol reacts with the functional group and an exchange occurs where a proton is transferred to the nitrogen and an ionic bond is formed to the anionic site of the resin. Contact is maintained until a threshold level is reached i.e. the breakthrough concentration. At this breakthrough point, the process reaches an equilibrium where additional organically bound nitrogen cannot be removed effectively. The flow is halted and the column is backwashed with water, preferably deionized or softened water. By flowing in reverse, the resin is fluidized and solids captured by the beads are loosened and removed.

In another embodiment, the solid adsorbent is a silica-alumina hydrate. Numerous silica-alumina catalyst compositions and processes for their preparation are described in the patent literature, see, e.g., U.S. Pat. No. 4,499,197.

Preferably, the alumina content of the silica-alumina hydrate is from about 10 to about 90 wt.-% of Al₂O₃. The preferred range of alumina content is from about 30 to about 70 wt.-% of Al₂O₃.

The introduction of silicon dioxide into aluminum oxide leads to the introduction of acidic centers. The number of acidic centers can be controlled by the amount of introduced silicon dioxide. The number of acidic centers increases with the amount of introduced silicon dioxide up to a maximum number of acidic centers, and decreases again with a further increasing amount of silicon dioxide after having reached the maximum number of acidic centers.

Examples of commercially available silica-alumina hydrates are Siral® available from Sasol Germany Gmbh, Hamburg, Germany. Siral® is based on orthorhombic aluminum oxide hydroxide (boehmite; AlOOH) and doped with SiO₂. Various Siral® grades having different ratios of Al₂O₃ to SiO₂ are available: Siral 1 (Al₂O₃/SiO₂=99/1), Siral 5 (Al₂O₃/SiO₂=95/5), Siral 10 (Al₂O₃/SiO₂=90/10), Siral 20 (Al₂O₃/SiO₂=80/20), Siral 28M (Al₂O₃/SiO₂=72/28), Siral 30 (Al₂O₃/SiO₂=70/30), Siral 40 (Al₂O₃/SiO₂=60/40). Siral 40 is especially preferred.

In an embodiment, the (iso)prenol is passed over a bed of the weakly acidic solid adsorbent. Suitably, said step of “passing over a bed” denotes that a layer (“bed”) of the weakly acidic solid adsorbent is provided in a customary reaction vessel known to the skilled person which may preferably be equipped with a stirring device, e.g. in a stirred-tank reactor. The (iso)prenol is then introduced into the reaction vessel and guided through the same in a manner that it gets into contact with the weakly acidic solid adsorbent.

Alternatively, the weakly acidic solid adsorbent may be provided in a reaction tube, e.g. of a tubular reactor and the (iso)prenol then continuously flows through said reaction tube(s) while getting into contact with the weakly acidic solid adsorbent.

In an embodiment, the (iso)prenol comprises, after contacting the alcohol stream with a weakly acidic solid adsorbent, less than 2 ppm of organically bound nitrogen. Herein, “ppm” denotes wt.-ppm of compounds incorporating organically bound nitrogen, relative to the total weight of the (iso)prenol.

Suitably, the content of organically bound nitrogen in the (iso)prenol may be determined by Kjeldahl analysis. Alternatively, an oxidative combustion method with a chemiluminescence detector according to DIN 51444 may be used.

Citral is a useful intermediate for, e.g., menthol or linalool.

Menthol may be prepared from citral via a process comprising the steps of
catalytic hydrogenation of citral to obtain citronellal;
cyclization of citronellal to obtain isopulegol in the presence of at least one acidic catalyst; and
catalytic hydrogenation of isopulegol to obtain menthol.

The overall reaction sequence is illustrated by the reaction scheme below.

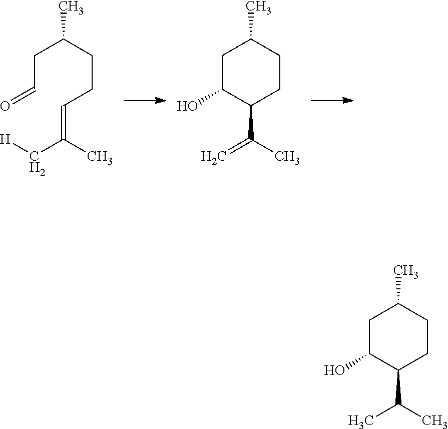

The hydrogenation of citral to obtain citronellal may be achieved by hydrogenation in the presence of a rhodium-phosphine catalyst.

The cyclization of citronellal to isopulegol may be achieved by cyclization in the presence of at least one Lewis-acidic aluminum-containing catalyst, such as a bis(diarylphenoxy)aluminum compound, which may be used in the presence of an auxiliary, such as a carboxylic anhydride. The isopulegol may be recovered from the catalyst-containing reaction product by distillative separation to give an isopulegol-enriched top product and an isopulegol-depleted bottom product. From the bottom product, the at least one catalyst may be regenerated. The isopulegol obtainable in this way by the cyclization of citronellal can be further purified by suitable separating and/or purification methods, in particular by crystallization, and be at least largely freed from undesired impurities or by-products.

The hydrogenation of isopulegol may be achieved by hydrogenation in the presence of at least one heterogeneous nickel-containing catalyst, preferably at least one heterogeneous nickel- and copper-containing catalyst.

Further details regarding the reaction sequence from citral to menthol may be found in US 2013/46118 A1, which is incorporated by reference herein.

In one aspect, the invention thus relates to an improved process for the preparation of menthol by producing citral using the above processes and then producing menthol from the citral. Menthol may be prepared as described herein or by other methods known in the art.

Linalool may be prepared from citral via a process comprising catalytic hydrogenation of citral to obtain nerol and/or geraniol, and isomerization thereof.

The hydrogenation of citral to obtain nerol and/or geraniol may be achieved by hydrogenation in the presence of at least one supported ruthenium, rhodium, osmium, iridium or platinum catalyst, preferably at least one ruthenium catalyst supported on carbon black.

The isomerization of nerol and/or geraniol to obtain linalool may be achieved by isomerization in the presence of at least one tungsten catalyst, in particular a dioxotungsten (VI) complex. Further details regarding the isomerization of nerol and/or geraniol may be found in U.S. Pat. No. 7,126,033 B2.

In one aspect, the invention thus relates to an improved process for the preparation of linalool by producing citral using the above processes and then producing linalool from the citral. Linalool may be prepared as described herein or by other methods known in the art.

The invention is further illustrated by the examples that follow.

### EXAMPLES

In the examples, the term “by-product(s)” is abbreviated with “BP”. With respect to GC-analysis, all “%” are reported as “area-%”.

### Example 1

This example illustrates the conversion and the formation of by-products in a model reaction mimicking the cleaving reaction of diprenyl acetal. Conversion and by-products are reported as a function of time at various combinations of cleaving temperature and catalyst concentration.

Diprenyl acetal (25 g, purity 73.8%, containing 1.6% 2,4,4-trimethyl-3-formyl-1,5-hexadiene, 5.4% prenyl (3-methylbutadienyl) ether, 5.8% BP 1a/b) was mixed with H₃PO₄ (85%) in an amount of 250 ppm, 530 ppm, or 750 ppm, respectively, and divided up equally into 6 microwave vessel (vol. 10 mL). The microwave vessels were sealed with a cap and heated to a temperature of 150 °C, 160 °C, 170 °C, or 180 °C, respectively. The individual microwave vessels were heated for a run time of 0 min, 5 min, 10 min, 15 min, 30 min and 60 min. After subsequent cooling to room temperature, solid K₂CO₃ (100-200 mg) was added to each reaction mixture and the reaction mixture was filtered using a syringe filter. Afterwards, the neutralized reaction mixtures were analyzed with GC. The results are reported in tables 1 to 7.

TABLE 1
Temperature: 150 °C, catalyst concentration: 530 ppm H₃PO₄.

| | time | prenol | prenal | [1] | [2] | [3] | citral | BP 1 | BP 2 | BP 4 | BP 5 | Σ citral-bb ^{[4]} | con. ^{[5]} of [1] | y. citral-bb ^{[6]} |
|---|---|---|---|---|---|---|---|---|---|---|---|---|---|---|
| run | [min] | [%] | [%] | [%] | [%] | [%] | [%] | [%] | [%] | [%] | [%] | [%] | [%] | [%] |
| 1-1 | 0 | 4.5 | 0.4 | 73.8 | 5.4 | 1.6 | 0.3 | 5.8 | 0.0 | 0.1 | 0.0 | 7.3 | 0 | 0.7 |
| 1-2 | 5 | 13.0 | 0.9 | 50.1 | 12.3 | 6.7 | 2.0 | 5.9 | 0.0 | 0.2 | 0.0 | 20.9 | 32 | 29.6 |
| 1-3 | 10 | 15.0 | 1.1 | 43.9 | 11.9 | 8.7 | 3.6 | 6.0 | 0.1 | 0.6 | 0.0 | 24.2 | 41 | 36.6 |
| 1-4 | 15 | 17.4 | 1.6 | 37.1 | 11.5 | 10.1 | 5.6 | 6.1 | 0.1 | 1.1 | 0.0 | 27.1 | 50 | 42.8 |
| 1-5 | 30 | 22.7 | 3.7 | 20.9 | 8.1 | 10.4 | 12.3 | 6.5 | 0.2 | 3.8 | 0.1 | 30.8 | 72 | 50.6 |
| 1-6 | 60 | 29.6 | 7.0 | 3.0 | 2.2 | 4.2 | 25.5 | 7.5 | 0.2 | 3.1 | 2.5 | 31.9 | 96 | 53.0 |

TABLE 2
Temperature: 160 °C, catalyst concentration: 530 ppm H₃PO₄.

| | time | prenol | prenal | [1] | [2] | [3] | citral | BP 1 | BP 2 | BP 4 | BP 5 | Σ citral-bb ^{[4]} | con. ^{[5]} of [1] | y. citral-bb ^{[6]} |
|---|---|---|---|---|---|---|---|---|---|---|---|---|---|---|
| run | [min] | [%] | [%] | [%] | [%] | [%] | [%] | [%] | [%] | [%] | [%] | [%] | [%] | [%] |
| 2-1 | 0 | 4.5 | 0.4 | 73.8 | 5.4 | 1.6 | 0.3 | 5.8 | 0.0 | 0.1 | 0.0 | 7.3 | 0 | 0.7 |
| 2-2 | 5 | 15.9 | 1.5 | 40.3 | 10.7 | 10.1 | 5.0 | 6.0 | 0.1 | 1.6 | 0.0 | 25.8 | 45 | 40.1 |
| 2-3 | 10 | 18.4 | 2.1 | 32.7 | 9.6 | 11.0 | 8.1 | 6.1 | 0.1 | 2.6 | 0.0 | 28.6 | 56 | 46.0 |
| 2-4 | 15 | 23.8 | 3.0 | 17.1 | 7.4 | 10.0 | 19.1 | 6.4 | 0.1 | 2.4 | 0.1 | 36.5 | 77 | 62.7 |
| 2-5 | 30 | 28.8 | 6.3 | 2.8 | 2.4 | 5.5 | 27.3 | 7.2 | 0.1 | 3.8 | 1.3 | 35.1 | 96 | 59.7 |
| 2-6 | 60 | 28.9 | 6.0 | 0.2 | 0.7 | 0.5 | 33.4 | 7.3 | 0.1 | 0.7 | 8.8 | 34.6 | 100 | 58.6 |

TABLE 3
Temperature: 170 °C, catalyst concentration: 530 ppm H₃PO₄.

| | time | prenol | prenal | [1] | [2] | [3] | citral | BP 1 | BP 2 | BP 4 | BP 5 | Σ citral-bb ^{[4]} | con. ^{[5]} of [1] | y. citral-bb ^{[6]} |
|---|---|---|---|---|---|---|---|---|---|---|---|---|---|---|
| run | [min] | [%] | [%] | [%] | [%] | [%] | [%] | [%] | [%] | [%] | [%] | [%] | [%] | [%] |
| 3-1 | 0 | 4.5 | 0.4 | 73.8 | 5.4 | 1.6 | 0.3 | 5.8 | 0.0 | 0.1 | 0.0 | 7.3 | 0 | 0.7 |
| 3-2 | 5 | 19.6 | 2.5 | 29.0 | 10.4 | 11.3 | 8.0 | 6.1 | 0.1 | 2.9 | 0.0 | 29.8 | 61 | 48.4 |
| 3-3 | 10 | 26.2 | 5.6 | 8.9 | 5.8 | 10.0 | 17.2 | 6.6 | 0.1 | 6.3 | 0.1 | 33.0 | 88 | 55.2 |
| 3-4 | 15 | 28.8 | 5.8 | 2.5 | 2.9 | 6.6 | 28.0 | 6.9 | 0.1 | 3.5 | 0.7 | 37.6 | 97 | 64.9 |
| 3-5 | 30 | 28.1 | 5.2 | 0.4 | 0.4 | 0.6 | 37.0 | 7.1 | 0.1 | 1.5 | 7.1 | 37.9 | 99 | 65.7 |
| 3-6 | 60 | 25.8 | 4.7 | 0.1 | 0.2 | 0.1 | 33.5 | 7.3 | 0.1 | 0.6 | 14.0 | 33.8 | 100 | 56.9 |

TABLE 4
Temperature: 170 °C, catalyst concentration: 250 ppm H₃PO₄.

| | time | prenol | prenal | [1] | [2] | [3] | citral | BP 1 | BP 2 | BP 4 | BP 5 | Σ citral-bb ^{[4]} | con. ^{[5]} of [1] | y. citral-bb ^{[6]} |
|---|---|---|---|---|---|---|---|---|---|---|---|---|---|---|
| run | [min] | [%] | [%] | [%] | [%] | [%] | [%] | [%] | [%] | [%] | [%] | [%] | [%] | [%] |
| 4-1 | 0 | 4.5 | 0.4 | 73.8 | 5.4 | 1.6 | 0.3 | 5.8 | 0.0 | 0.1 | 0.0 | 7.3 | 0 | 0.7 |
| 4-2 | 5 | 17.0 | 0.8 | 41.9 | 8.4 | 9.7 | 6.6 | 6.2 | 0.0 | 0.4 | 0.0 | 24.6 | 43 | 37.5 |
| 4-3 | 10 | 22.1 | 1.5 | 30.6 | 6.7 | 9.2 | 12.4 | 6.7 | 0.1 | 1.3 | 0.0 | 28.2 | 59 | 45.1 |
| 4-4 | 15 | 27.7 | 2.8 | 17.3 | 4.9 | 7.7 | 18.1 | 7.4 | 0.1 | 3.3 | 0.3 | 30.7 | 77 | 50.4 |
| 4-5 | 30 | 27.9 | 4.4 | 1.8 | 0.7 | 1.1 | 40.2 | 6.7 | 0.1 | 3.3 | 3.6 | 41.9 | 98 | 74.2 |
| 4-6 | 60 | 27.3 | 4.3 | 0.2 | 0.1 | 0.1 | 39.3 | 6.8 | 0.1 | 0.8 | 10.4 | 39.4 | 100 | 68.9 |

TABLE 5
Temperature: 180 °C, catalyst concentration: 530 ppm H₃PO₄.

| | time | prenol | prenal | [1] | [2] | [3] | citral | BP 1 | BP 2 | BP 4 | BP 5 | Σ citral-bb ^{[4]} | con. ^{[5]} of [1] | y. citral-bb ^{[6]} |
|---|---|---|---|---|---|---|---|---|---|---|---|---|---|---|
| run | [min] | [%] | [%] | [%] | [%] | [%] | [%] | [%] | [%] | [%] | [%] | [%] | [%] | [%] |
| 5-1 | 0 | 4.5 | 0.4 | 73.8 | 5.4 | 1.6 | 0.3 | 5.8 | 0.0 | 0.1 | 0.0 | 7.3 | 0 | 0.7 |
| 5-2 | 5 | 26.2 | 3.1 | 7.7 | 4.2 | 8.0 | 31.2 | 6.5 | 0.1 | 2.7 | 0.3 | 43.4 | 90 | 77.2 |
| 5-3 | 10 | 28.2 | 4.5 | 0.8 | 0.8 | 2.4 | 39.3 | 6.9 | 0.1 | 3.2 | 2.3 | 42.6 | 99 | 75.5 |
| 5-4 | 15 | 28.0 | 4.2 | 0.3 | 0.3 | 0.4 | 41.4 | 6.9 | 0.1 | 1.2 | 5.8 | 42.2 | 100 | 74.7 |
| 5-5 | 30 | 26.5 | 4.2 | 0.1 | 0.2 | 0.1 | 37.9 | 7.0 | 0.1 | 0.5 | 11.2 | 38.2 | 100 | 66.3 |
| 5-6 | 60 | 23.7 | 3.7 | 0.0 | 0.1 | 0.0 | 34.3 | 7.2 | 0.0 | 0.5 | 16.1 | 34.5 | 100 | 58.3 |

TABLE 6
Temperature: 180 °C, catalyst concentration: 250 ppm H₃PO₄.

| | time | prenol | prenal | [1] | [2] | [3] | citral | BP 1 | BP 2 | BP 4 | BP 5 | Σ citral-bb ^{[4]} | con. ^{[5]} of [1] | y. citral-bb ^{[6]} |
|---|---|---|---|---|---|---|---|---|---|---|---|---|---|---|
| run | [min] | [%] | [%] | [%] | [%] | [%] | [%] | [%] | [%] | [%] | [%] | [%] | [%] | [%] |
| 6-1 | 0 | 4.5 | 0.4 | 73.8 | 5.4 | 1.6 | 0.3 | 5.8 | 0.0 | 0.1 | 0.0 | 7.3 | 0 | 0.7 |
| 6-2 | 5 | 23.6 | 1.5 | 27.6 | 7.1 | 9.2 | 13.6 | 6.8 | 0.1 | 1.0 | 0.0 | 29.9 | 63 | 48.6 |
| 6-3 | 10 | 27.0 | 3.3 | 4.4 | 1.2 | 2.9 | 41.2 | 6.7 | 0.1 | 3.0 | 1.2 | 45.3 | 94 | 81.3 |
| 6-4 | 15 | 28.3 | 3.7 | 1.3 | 0.7 | 1.1 | 43.7 | 6.7 | 0.1 | 2.2 | 2.9 | 45.4 | 98 | 81.5 |
| 6-5 | 30 | 27.6 | 3.8 | 0.1 | 0.1 | 0.1 | 42.4 | 6.7 | 0.1 | 1.0 | 7.8 | 42.6 | 100 | 75.7 |
| 6-6 | 60 | 25.6 | 3.5 | 0.0 | 0.1 | 0.0 | 38.5 | 6.8 | 0.0 | 0.4 | 12.7 | 38.6 | 100 | 67.0 |

TABLE 7
Temperature: 160 °C, catalyst concentration: 750 ppm H₃PO₄.

| | time | prenol | prenal | [1] | [2] | [3] | citral | BP 1 | BP 2 | BP 4 | BP 5 | Σ citral-bb ^{[4]} | con. ^{[5]} of [1] | y. citral-bb ^{[6]} |
|---|---|---|---|---|---|---|---|---|---|---|---|---|---|---|
| run | [min] | [%] | [%] | [%] | [%] | [%] | [%] | [%] | [%] | [%] | [%] | [%] | [%] | [%] |
| 7-1 | 0 | 4.5 | 0.4 | 73.8 | 5.4 | 1.6 | 0.3 | 5.8 | 0.0 | 0.1 | 0.0 | 7.3 | 0 | 0.7 |
| 7-2 | 5 | 16.4 | 1.2 | 39.0 | 10.6 | 10.8 | 6.3 | 6.1 | 0.1 | 0.8 | 0.0 | 27.6 | 47 | 43.9 |
| 7-3 | 10 | 20.1 | 1.8 | 28.0 | 7.8 | 11.6 | 13.5 | 6.3 | 0.1 | 1.5 | 0.0 | 33.0 | 62 | 55.2 |
| 7-4 | 15 | 23.1 | 3.3 | 17.4 | 5.6 | 10.4 | 19.0 | 6.6 | 0.2 | 4.1 | 0.2 | 35.1 | 76 | 59.6 |
| 7-5 | 30 | 28.2 | 6.5 | 2.2 | 1.0 | 3.3 | 29.6 | 7.5 | 0.2 | 4.6 | 3.4 | 33.9 | 97 | 57.1 |
| 7-6 | 60 | 27.4 | 6.3 | 0.4 | 0.3 | 0.3 | 30.7 | 7.7 | 0.1 | 1.2 | 12.5 | 31.2 | 100 | 51.5 |

[1] diprenyl acetal
[2] prenyl (3-methylbutadienyl) ether
[3] 2,4,4-trimethyl-3-formyl-1,5-hexadiene
^{[4]} sum of amount of citral building blocks = prenyl (3-methyl-butadienyl) ether + 2,4,4-trimethyl-3-formyl-1,5-hexadiene + citral
^{[5]} conversion
^{[6]} yield of citral building blocks = prenyl (3-methyl-butadienyl) ether + 2,4,4-trimethyl-3-formyl-1,5-hexadiene + citral

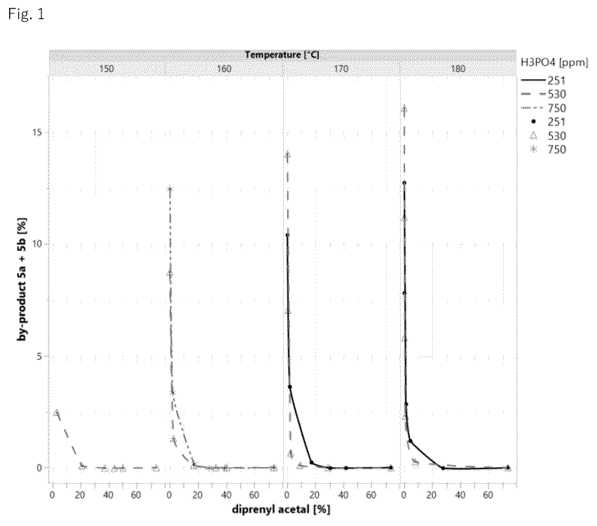

The evolution of by-product 5 vs. the residual concentration of diprenyl acetal for each of the tested temperature and catalyst concentration (see tables 1 to 7) is depicted in FIG. 1. It can be seen from FIG. 1 that for all temperatures and catalyst concentrations, the formation of by-product 5 rises sharply as the content of diprenyl acetal approaches 0%, i.e. the conversion approaches 100%.

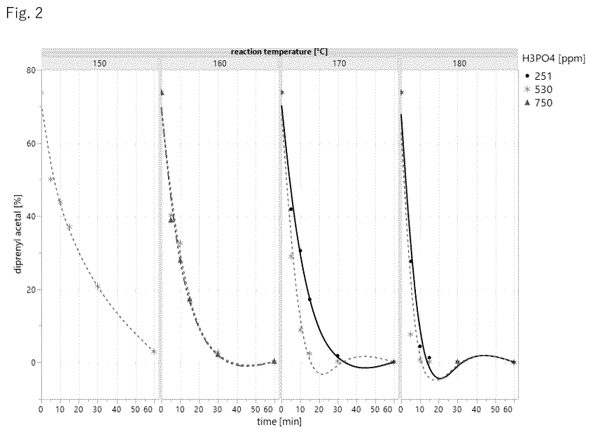

FIG. 2 depicts the concentration of diprenyl acetal over time for each of the tested temperature and catalyst concentration (see tables 1 to 7). It can be seen that the higher temperatures and the higher the catalyst concentration, the faster the reaction proceeds.

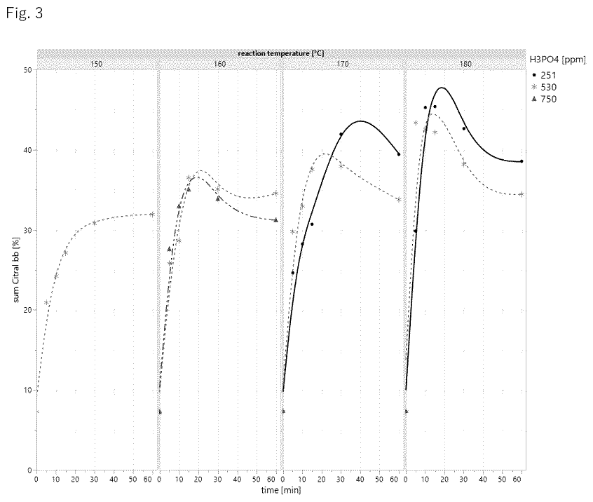

FIG. 3 depicts the concentration of Σcitral building blocks (prenyl (3-methyl-butadienyl) ether, 2,4,4-trimethyl-3-formyl-1,5-hexadiene and citral) over time for each of the tested temperature and catalyst concentration (see tables 1 to 7). FIG. 3 shows an initial increase of the concentration of Σcitral building blocks. For the runs at temperatures of 160 °C or higher, the content of Σcitral building blocks decreases after a maximum is reached.

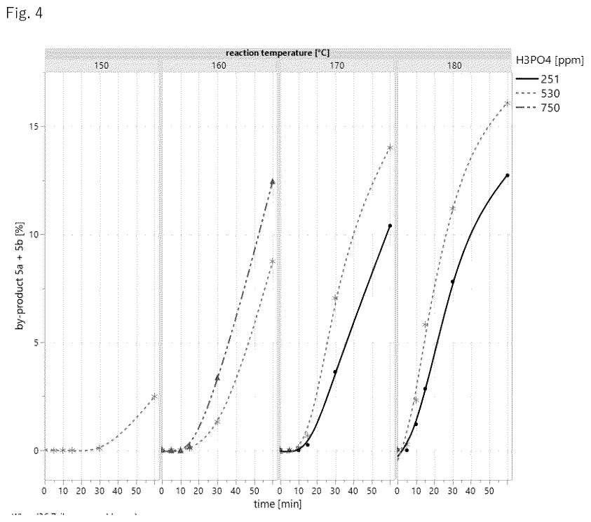

FIG. 4 depicts the concentration of by-product 5 over time for each of the tested temperature and catalyst concentration (see tables 1 to 7). It can be seen from FIG. 4 that initially, the formation of by-product 5 is slow, followed by a sharp increase of the concentration of by-product 5. It can also be taken from FIG. 4 that the formation of by-product 5 commences earlier and is steeper with increasing cleaving temperature and concentration of phosphoric acid.

### Example 2

Example 2 illustrates the continuous reactive distillation of diprenyl acetal with continuous withdrawal of the cleaving fraction (prenyl (3-methyl-butadienyl) ether, 2,4,4-trimethyl-3-formyl-1,5-hexadiene and citral).

A rectification column having a height of 800 mm with structured packings (Montz A3-750) equipped with an external evaporator and a circulation pump having a hold up of 240 mL and a top condenser and a reflux divider was used.

The column was operated at a top pressure of 50 mbar. The feed stream of diprenyl acetal (content 75%) was mixed with the sump stream and dosed on the evaporator.

The H₃PO₄-catalyst was diluted in prenol and dosed as a 1%-solution into the sump circulatory stream. For every experiment, a sufficient lead time of 5-6 h was chosen until steady state conditions were obtained. In runs 8-1 to 8-4, a distillate stream was taken overhead. In runs 8-5 to 8-6, a side draw and a head stream were taken. Samples of the sump and distillate streams were taken and analyzed with GC. The accurate H₃PO₄ concentration in the sump was determined via elemental P-analysis. The reaction conditions (residence time, amount of H₃PO₄ and temperature) are shown in tables 8a and 8b.

TABLE 8a

| | | | | | | | distillate stream | | | | | sump stream | | |
|---|---|---|---|---|---|---|---|---|---|---|---|---|---|---|
| | conditions | | | | | | | | Σ citral- | con. ^{[5]} | y. citral- | | y. BP | y. BP |
| | feed ^{[0]} | time | H₃PO₄ | H₃PO₄ | T | [3] | flow | [3] | bb ^{[4]} | of [3] | bb ^{[6]} | flow | 2 ^{[7]} | 5 ^{[8]} |
| run | [g/h] | [h] | [ppm] ^{[1]} | [ppm] ^{[2]} | [°C] | [%] | [g/h] | [g/h] | [g/h] | [%] | [%] | [g/h] | [%] | [%] |
| 8-1 | 305 | 0.9 | 34 | 664 | 157 | 74.3 | 289 | 12.8 | 134.6 | 94 | 93 | 15.5 | 0.01 | 0.60 |
| 8-2 | 305 | 0.9 | 34 | 664 | 159 | 75.0 | 289 | 14.0 | 132.9 | 95 | 91 | 15.4 | 0.07 | 0.60 |
| 8-3 | 305 | 0.9 | 34 | 882 | 171 | 75.3 | 292 | 12.9 | 132.0 | 94 | 90 | 12.3 | 0.03 | 0.43 |
| 8-4 | 305 | 0.9 | 49 | 1138 | 173 | 75.2 | 291 | 13.5 | 126.0 | 94 | 86 | 12.9 | 0.03 | 0.45 |

TABLE 8b

| | conditions | | | | | |
|---|---|---|---|---|---|---|
| | feed ^{[0]} | time | H₃PO₄ | H₃PO₄ | T | [3] |
| run | [g/h] | [h] | [ppm] ^{[1]} | [ppm] ^{[2]} | [°C] | [%] |
| 8-5 | 204 | 1.4 | 35 | 885 | 154 | 78.8 |
| 8-6 | 204 | 1.4 | 35 | 916 | 152 | 75.1 |

| | Distillate streams | | | | | | | | | | |
|---|---|---|---|---|---|---|---|---|---|---|---|
| | Head stream | | | | Side draw | | | | | | |
| | flow | Prenol [GC- | BP 1b | [3] | flow | Prenol [GC- | BP 1b | [3] | Σ citral-bb ^{[4]} | con. ^{[5]} of [3] | y. citral-bb ^{[6]} |
| run | [g/h] | wt-%] | [GC-wt. %] | [wt.-%] | [g/h] | wt-%] | [GC-wt. %] | [wt.-%] | [g/h] | [%] | [%] |
| 8-5 | 101 | 60.5 | 10.5 | 0.14 | 91.5 | 0.83 | 0.88 | 4.7 | 99.6 | 97 | 97 |
| 8-6 | 101 | 54.2 | 8.7 | 0 | 78.9 | 1.06 | 0.85 | 3.74 | 96.9 | 98 | 99 |

| | sump stream | | |
|---|---|---|---|
| | flow | y. BP 2 ^{[7]} | y. BP 5 ^{[8]} |
| run | [g/h] | [%] | [%] |
| 8-5 | 9.1 | 0.04 | 0.47 |
| 8-6 | 7.7 | 0.03 | 0.35 |

^{[0]} containing 75% of diprenyl acetal, 5.6% of by-product 1b, 4% of prenol
^{[1]} H₃PO₄ content in the feed
^{[2]} H₃PO₄ content in the sump of the distillation column.
[3] diprenyl acetal
^{[4]} sum of amount of citral building blocks = prenyl (3-methyl-butadienyl) ether + 2,4,4-trimethyl-3-formyl-1,5-hexadiene + citral
^{[5]} conversion
^{[6]} yield of citral building blocks = prenyl (3-methyl-butadienyl) ether + 2,4,4-trimethyl-3-formyl-1,5-hexadiene + citral
^{[7]} yield of by-product 2 (BP 2)
^{[8]} yield of by-product 5 (BP 5)

Tables 8a and 8b show higher yields of citral building blocks with less by-product formation in comparison to example 1. Comparison of runs 8-2, 8-3, 8-4 and 8-5 indicates that a balance between the concentration of H₃PO₄ and reaction temperature must be maintained. Too high a concentration of H₃PO₄ at high temperatures may be detrimental with respect to the yield of citral building blocks.

### Example 3

Example 3 illustrates the formation of by-products BP 1a/b, BP 2, BP 3, BP 4 and BP 5a/b if the intermediate 2,4,4-trimethyl-3-formyl-1,5-hexadiene is carried over to step a) and subjected to the reaction conditions of steps a). The example also elucidates the fate in step b) of 2,4,4-trimethyl-3-formyl-1,5-hexadiene and the products formed therefrom in step a).

In a 500 mL reactor equipped with a 15 cm Vigreux distillation column, a condenser, a distillation template and a vacuum pump, 2,4,4-trimethyl-3-formyl-1,5-hexadiene (176 g, purity 92%, containing 6% citral) was dissolved in prenol (208 g, 2.4 mol) and cyclohexane (300 mL). After addition of HNO₃ (500 ppm), the reaction mixture was refluxed (80-95 °C, 200 mbar) and water formed during the reaction was removed via azeotropic distillation using a Dean-Stark apparatus. After refluxing for 3 h, 13 g of water was removed from the reaction mixture. The reaction mixture was analyzed via GC. The results are shown in table 9.

TABLE 9

| prenol | BP 1a/b ^{[1]} | [2] | BP 2 | BP 3 | BP 4 | BP 5 | citral |
|---|---|---|---|---|---|---|---|
| [wt.-%] | [wt.-%] | [wt.-%] | [wt.-%] | [wt.-%] | [wt.-%] | [wt.-%] | [wt.-%] |
| 20.0 | 10.0 | 21.0 | 26.9 | 10.1 | 0.7 | n.d. ^{[3]} | 4.0 |

^{[1]} ratio 1:1
[2] 2,4,4-trimethyl-3-formyl-1,5-hexadiene
^{[3]} n.d. = not determined

The vacuum was gradually decreased to 1 mbar and the low boiling compounds (HNO₃, cyclohexane, prenol, prenal, BP 1a/b, citral) were distilled from the reaction mixture. 103 g of a yellowish sump residue was obtained. The sump was analyzed via GC. The results are shown in table 10.

TABLE 10

| prenol | BP 1a/b | [1] | BP 2 | BP 3 | BP 4 | BP 5 | citral |
|---|---|---|---|---|---|---|---|
| [wt.-%] | [wt.-%] | [wt.-%] | [wt.-%] | [wt.-%] | [wt.-%] | [wt.-%] | [wt.-%] |
| n.d. ^{[2]} | n.d. ^{[2]} | n.d. ^{[2]} | 75.0 | 14.7 | 3.2 | n.d. ^{[2]} | 0.8 |

[1] 2,4,4-trimethyl-3-formyl-1,5-hexadiene
^{[2]} n.d. = not determined

The catalyst H₃PO₄ (85%, 0.03 g) was added to the residue and at a vacuum of mbar, the temperature was gradually increased to 140-145 °C. During temperature increase, the formed products were distilled from the reaction mixture. After 2 h, 55 g of distillate was collected. The distillate was analyzed via GC. The results are shown in table 11.

TABLE 11

| prenol | BP 1a/b | [1] | BP 2 | BP 3 | BP 4 | BP 5 | citral |
|---|---|---|---|---|---|---|---|
| [wt.-%] | [wt.-%] | [wt.-%] | [wt.-%] | [wt.-%] | [wt.-%] | [wt.-%] | [wt.-%] |
| 20.1 | n.d. ^{[2]} | n.d. ^{[2]} | 0.75 | 74.7 | n.d. ^{[2]} | 0.85 | 0.8 |

[1] 2,4,4-trimethyl-3-formyl-1,5-hexadiene
^{[2]} n.d. = not determined

Example 3 evidences that under the reaction conditions of step a), 2,4,4-trimethyl-3-formyl-1,5-hexadiene is converted mostly to BP 2 and BP 3 (with prenol as side-product) with only small amounts of citral. The sump fraction remaining after removal of the low boiling compounds mainly consists of BP 2 and BP 3 (see table 10). It is considered that the sump fraction mimics the contaminants carried over together with the crude diprenyl acetal into step b). Under the reaction conditions of step b), mostly BP 3 is formed from BP 2 with prenol as side-product. No further citral is obtained from the sump fraction under the reaction conditions of step b). Hence, BP 3 cannot be converted to citral and can be considered as a dead end in the reaction route.

### Example 4

Example 4 illustrates the influence of the reaction temperature and the concentration of nitric acid in step a).

In a 250 mL three neck flask equipped with an oil-bath, magnetic stirrer, 15 cm Vigreux distillation column, condenser, distillation template and a vacuum pump, the starting materials prenol (120.4 g, 1.4 mol) and prenal (58.8 g, 0.7 mol) were added and stirred at room temperature. After addition of HNO₃ (65%) in an amount of 100 ppm, 250 ppm, or 500 ppm, respectively, the reaction mixture was evacuated to 100 mbar and the temperature was increased to 80-82 °C, or 90-91 °C, respectively, until the reaction mixture started to reflux. The formed water was removed via azeotropic distillation and the vacuum was decreased gradually to 70 mbar. After 1 h, the reaction was stopped. The sump and the organic phase in the water separator were analyzed via GC. The results are shown in table 12.

TABLE 12

| | | | | light boilers | | | | | | | | | | | | |
|---|---|---|---|---|---|---|---|---|---|---|---|---|---|---|---|---|
| | | | [0] | in water separator | | | sump | | | | | | conversion ^{[x]} | | selectivity ^{[y]} | |
| | HNO₃ | T | H₂O | m | prenol | prenal | m | prenol | prenal | [1] | BP 1 | Σ [2] | prenol | prenal | prenol | prenal |
| # | [ppm] | [°C] | [g] | [g] | [wt-%] | [wt-%] | [g] | [wt-%] | [wt-%] | [wt-%] | [wt-%] | [wt-%] | [%] | [%] | [%] | [%] |
| 9-1 | 100 | 80-82 | 5.2 | 6.5 | 21.4 | 59.3 | 167.5 | 37.6 | 14.8 | 44.0 | 0.4 | 0.14 | 46.5 | 49.8 | 95.8 | 92.2 |
| 9-2 | 250 | 80-81 | 5.8 | 7.1 | 32.1 | 60.9 | 166.3 | 32.1 | 12.1 | 50.2 | 1.1 | 0.73 | 54.5 | 57.1 | 91.8 | 90.2 |
| 9-3 | 500 | 80-82 | 5.5 | 6.7 | 21.5 | 56.0 | 167.0 | 33.3 | 13.4 | 43.7 | 1.4 | 1.70 | 52.7 | 55.3 | 83.1 | 81.5 |
| 9-4 | 500 | 90-91 | 5.7 | 6.6 | 21.1 | 47.8 | 166.9 | 36.7 | 17.0 | 29.9 | 6.1 | 3.50 | 47.9 | 44.8 | 60.8 | 68.9 |

[0] removed from water separator
[1] diprenyl acetal
[2] sum of amount of high-boiling by-products (BP 2 to BP 5)
^{[x]} conversion of prenol or prenal
^{[y]} selectivity to diprenyl acetal, relative to prenol or prenal

It can be seen from table 12 that an increase of the reaction temperature and the concentration of nitric acid in step a) results in increase of the formation of by-product 1 and high-boiling by-products (BP 2 to BP 5), and in a decrease of the selectivity of prenol and prenal.

1.-17. (canceled)

18. A process for the preparation of 3,7-dimethyl-octa-2,6-dienal (citral) comprising the steps of:
a) continuously condensing prenol with prenal in the presence of at least one catalyst in a reaction column with water of condensation being distilled off as a prenal-water azeotrope as a vapor, at least partially condensing the vapor and separating the condensate into an aqueous phase and an organic phase and directing the organic phase partially as a reflux to the reaction column and partially discharging the organic phase as a purge stream,
while continuously withdrawing an acetal fraction comprising diprenyl acetal of prenal from the reaction column,
wherein the reaction temperature is below 100 °C, the catalyst is nitric acid and the concentration of the nitric acid is below 500 ppm;
b) continuously subjecting the acetal fraction in a cleaving column to cleaving conditions in the presence of at least one catalyst with elimination of prenol while continuously withdrawing from the cleaving column a cleaving fraction containing at least one of prenyl (3-methyl-butadienyl) ether and 2,4,4-trimethyl-3-formyl-1,5-hexadiene, and optionally containing citral,
wherein the conversion rate of diprenyl acetal of prenal in step b) is maintained at above 90% and below 100%, and the unreacted diprenyl acetal is at least partially contained in the withdrawn cleaving fraction;
c) reacting the cleaving fraction in a plug-flow type reactor to obtain citral; and
d) recycling part of the prenol obtained in step b) to step a).

19. The process according to claim 18, wherein the cleaving temperature in step b) is above 150 °C and below 200 °C.

20. The process according to claim 18, wherein the catalyst in step b) is phosphoric acid.

21. The process according to claim 20, wherein the concentration of the phosphoric acid in the bottoms of the cleaving column is above 100 ppm and below 1500 ppm.

22. The process according to claim 18, wherein the residence time in step b) is above 5 min and below 90 min.

23. The process according to claim 18, wherein the cleaving fraction is withdrawn from the cleaving column as a side draw, and the eliminated prenol is withdrawn via the top of the cleaving column.

24. The process according to claim 18, wherein the rate of the purge stream is such that a total stationary concentration of by-products 1a and 1b 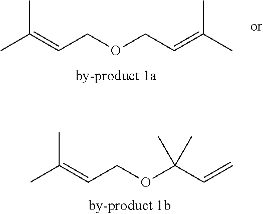
is maintained below 7 wt.-% in the feed to step a).

25. The process according to claim 18, wherein the reaction pressure in step a) is below 150 mbar.

26. The process according to claim 18, wherein the concentration of 2,4,4-trimethyl-3-formyl-1,5-hexadiene of the prenol recycled from step b) into step a) is controlled such that the concentration of 2,4,4-trimethyl-3-formyl-1,5-hexadiene in step a) is below 1 wt.-%, relative to the total weight of prenol and prenal.

27. The process according to claim 18, wherein the concentration of citral of the prenol recycled from step b) into step a) is controlled such that the concentration of citral in step a) is below 1 wt.-%, relative to the total weight of prenol and prenal.

28. The process according to claim 18, wherein prenol is obtained by reacting at least one formaldehyde source and isobutylene to obtain 3-methylbut-3-en-1-ol (isoprenol), and subjecting at least part of the obtained isoprenol to isomerization by bringing a reactant stream comprising isoprenol into contact with at least one heterogeneous isomerization catalyst.

29. The process according to claim 28, wherein the isoprenol is obtained by reacting the at least one formaldehyde source and isobutylene in a reactor to obtain isoprenol, wherein reacting the at least one formaldehyde source and isobutylene comprises at least one of α, β and γ:
α) mixing and injecting the at least one formaldehyde source and isobutylene into a reactor through a plurality of nozzles operated in parallel and reacting the formaldehyde source and isobutylene under supercritical conditions; wherein the reactor comprises a vertically disposed vessel, a sidewall, an upper portion and a lower portion; and wherein the formaldehyde source and isobutylene are injected into a mixing chamber of the reactor disposed in the upper portion and a fluid comprising formaldehyde and/or isobutylene and/or isoprenol is passed from the mixing chamber into a post-reaction chamber disposed in the lower portion; and
providing draft tubes arranged essentially concentrically underneath each of the nozzles in the mixing chamber, the draft tubes providing downcomer conduits within the draft tubes and a riser conduit outside of the draft tubes, so that the formaldehyde source and isobutylene injected through the nozzles travel generally downward in the downcomer conduits, a fluid comprising formaldehyde and/or isobutylene and/or isoprenol is then diverted in a generally upward direction in the riser conduit, and the fluid is back-mixed with the injected formaldehyde source and isobutylene;
β) mixing and injecting the at least one formaldehyde source and isobutylene into an internal loop reactor through at least one nozzle into first conduit(s), the internal loop reactor comprising:
a vertically disposed cylindrical vessel comprising a sidewall;
at least one draft tube having a tube inlet end and a tube outlet end, arranged vertically within the vessel, the draft tube(s) being arranged concentrically to the nozzle(s), and having an inner surface and an outer surface, wherein the draft tube(s) provide(s) the first conduit(s) within the draft tube(s), and a second conduit outside of the draft tube(s) and within the sidewall, the first conduit(s) being in fluid communication with the second conduit;
reactor fluid outlet means;
wherein the inner surface of the draft tube(s) convexly curves so that the first conduit(s) exhibit(s) an annular constriction of the cross-section between the tube inlet end and the tube outlet end; wherein the constriction is located closer to the tube inlet end; wherein the convex curvature of the inner surface of the draft tube(s) extends over at least 70% of the length of the draft tube; and
wherein the outer surface of the draft tube(s) convexly curves so that the draft tube(s) exhibit(s) a circumferential protuberance between the tube inlet end and the tube outlet end; wherein the convex curvature of the outer surface of the draft tube(s) extends over at least 70% of the length of the draft tube; and
wherein the edges of the draft tube(s) are rounded so that the at least one formaldehyde source and isobutylene injected through the nozzles travel generally downward in the first conduit(s) to obtain a reacted fluid, the reacted fluid is then diverted in the opposite direction so as to travel through the second conduit and is subsequently back-mixed with the injected fluid;
γ) heat-exchanging a stream of hot isoprenol withdrawn from the reactor with an isobutylene stream directed to the reactor;
wherein heat-exchanging is performed in at least two shell-and-tube heat exchangers;
each of the heat exchangers comprising a plurality of tubes and a shell-side heat exchange passage;
wherein the hot isoprenol is directed through the tubes of the heat exchangers; and the isobutylene is guided through the shell-side passage, and at least two of the heat exchangers are connected in series with regard to both the shell-side flow and the tube-side flow.

30. The process according to claim 28, additionally comprising at least one of αα and ββ:
αα) purification of isoprenol by subjecting a stream of crude isoprenol containing isoprenol, water and formaldehyde, or an isoprenol containing fraction thereof, to distillation in a low-boiler separation tower operated at a pressure of 2 bara or higher to obtain a distillate stream containing aqueous formaldehyde and a bottoms stream containing isoprenol essentially free of formaldehyde;
ββ) maintaining in the reactant stream a weight ratio of formaldehyde to isoprenol of less than 0.04.

31. The process according to claim 18, wherein prenal is provided by at least one of γ-i) and γ-ii):
γ-i) subjecting isoprenol to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal;
wherein step γ-i) is optionally characterized by maintaining in the reactant stream a weight ratio of formaldehyde to isoprenol of less than 0.04;
γ-ii) oxidizing prenol so as to obtain prenal by bringing a reactant stream comprising prenol into contact with at least one oxidant and at least one oxidation catalyst.

32. The process according to claim 31, wherein oxidative dehydrogenation of step γ-i) is carried out by passing the isoprenol through a plurality of reaction tubes of a shell-and-tube heat exchange reactor comprising
a shell-side heat exchange passage for circulating a heat transfer medium and a reaction passage comprising the plurality of reaction tubes;
an inlet for introducing the reactant stream to the reaction passage; and
an outlet from the reaction passage for recovering an effluent stream from the reaction tubes;
wherein the reaction tubes comprise:
a reactant pre-heating zone adjacent to the inlet, and
a reaction zone downstream of the reactant pre-heating zone, the reaction zone having a catalytically active wire matrix insert having silver at least on a part of its surface.

33. The process according to claim 32, wherein the catalytically active wire matrix inserts comprise an elongated core having a plurality of wire loops extending from the elongated core, wherein the wire loops are longitudinally arranged and helically shifted, and wherein the wire loops comprise a massive silver wire.

34. A process for the preparation of a citral-derived chemical, comprising preparing citral by the process according to claim 18, and at least one of ααα, βββ or (βββ plus γγγ):
ααα) converting the citral to obtain menthol;
βββ) converting the citral to geraniol and/or nerol;
γγγ) converting the geraniol and/or nerol to obtain linalool.
